(19) United States
(12) Patent Application Publication
(10) Pub. No.: US 2017/0280988 A1
(43) Pub. Date: Oct. 5, 2017

(54) DEVICES AND METHODS FOR MINIMALLY INVASIVE SURGERY

(71) Applicant: VisionScope Technologies LLC, Littleton, MA (US)

(72) Inventors: Louis J. BARBATO, Franklin, MA (US); Gregg E. FAVALORA, Bedford, MA (US); Hjalmar POMPE VAN MEERDERVOORT, Boston, MA (US); Thomas J. GILL, IV, Weston, MA (US)

(21) Appl. No.: 15/508,845

(22) PCT Filed: Sep. 3, 2015
(86) PCT No.: PCT/US2015/048428

§ 371 Date: Mar. 3, 2017

Related U.S. Application Data
(63) Continuation-in-part of application No. 14/677,895, filed on Apr. 2, 2015.
(60) Provisional application No. 62/045,490, filed on Sep. 3, 2014.
(60) Provisional application No. 62/003,287, filed on May 27, 2014.
(60) Provisional application No. 61/979,476, filed on Apr. 14, 2014.
(60) Provisional application No. 61/974,427, filed on Apr. 2, 2014.

(51) Int. Cl. A61B 1/317; A61B 17/16; A61B 1/313; A61B 1/04; A61B 1/06; A61B 1/07; A61B 17/00; A61B 1/018

(52) U.S. Cl. CPC A61B 1/317; A61B 17/00234; A61B 17/16; A61B 1/018; A61B 1/042; A61B 1/0684; A61B 1/07; A61B 1/3132; A61B 2017/00221; A61B 2017/003; A61B 2017/00336; A61B 2017/4216

(57) ABSTRACT

The present invention relates to methods and devices for minimally invasive diagnosis and treatment of joint injuries and other injuries or diseases in body joints, tissues, and cavities. Small diameter endoscopic devices are used for visualization and to provide access for the insertion of small diameter surgical tools without the use of distending fluid. Preferred embodiments of the endoscopic devices can utilize wireless transmission to a handheld display device to visualize diagnostic and therapeutic procedures in accordance with the invention.

### CROSS-REFERENCE TO RELATED APPLICATIONS

This application claims priority to U.S. Provisional Application No. 61/974,427 filed Apr. 2, 2014, U.S. Provisional Application No. 61/979,476 filed Apr. 14, 2014, U.S. Provisional Application No. 62/003,287 filed May. 27, 2014, U.S. Provisional Application No. 62/045,490 filed Sep. 3, 2014, and U.S. patent application Ser. No. 14/677,895 filed Apr. 2, 2015, the entire contents of these applications being incorporated herein by reference.

### BACKGROUND OF THE INVENTION

The medial meniscus and lateral meniscus are crescent-shaped bands of thick, pliant cartilage attached to the shinbone (fibia). Meniscectomy is the surgical removal of all or part of a torn meniscus. The lateral meniscus is on the outside of the knee, is generally shaped like a circle, and covers 70% of the tibial plateau. The medial meniscus is on the inner side of the knee joint, has a C shape, and is thicker posteriorly. As the inner portion of the meniscus does not have good vascular flow, tears are less likely to heal. The current surgical procedure for treating damaged meniscus cartilage typically involves partial meniscectomy by arthroscopic removal of the unstable portion of the meniscus and balancing of the residual meniscal rim. Postoperative therapy typically involves treatment for swelling and pain, strengthening exercises, and limits on the level of weight bearing movement depending on the extent of tissue removal.

Existing arthroscopic techniques utilize a first percutaneous entry of an arthroscope that is 4-5 mm in diameter to inspect the condition of the meniscus. After visual confirmation as to the nature of the injury, the surgeon can elect to proceed with insertion of surgical tools to remove a portion of the meniscus.

A hip joint is essentially a ball and socket joint. It includes the head of the femur (the ball) and the acetabulum (the socket). Both the ball and socket are congruous and covered with hyaline cartilage (hyaline cartilage on the articular surfaces of bones is also commonly referred to as articular cartilage), which enables smooth, almost frictionless gliding between the two surfaces. The edge of the acetabulum is surrounded by the acetabular labrum, a fibrous structure that envelops the femoral head and forms a seal to the hip joint. The acetabular labrum includes a nerve supply and as such may cause pain if damaged. The underside of the labrum is continuous with the acetabular articular cartilage so any compressive forces that affect the labrum may also cause articular cartilage damage, particularly at the junction between the two (the chondrolabral junction).

The acetabular labrum may be damaged or torn as part of an underlying process, such as Femoroacetabular impingement (FAI) or dysplasia, or may be injured directly by a traumatic event. Depending on the type of tear, the labrum may be either trimmed (debrided) or repaired. Various techniques are available for labral repair that mainly use anchors, which may be used to re-stabilise the labrum against the underlying bone to allow it to heal in position.

Similarly, articular cartilage on the head of femur and acetabulum may be damaged or torn, for example, as a result of a trauma, a congenital condition, or just constant wear and tear. When articular cartilage is damaged, a torn fragment may often protrude into the hip joint causing pain when the hip is flexed. Moreover, the bone material beneath the surface may suffer from increased joint friction, which may eventually result in arthritis if left untreated. Articular cartilage injuries in the hip often occur in conjunction with other hip injuries and like labral tears.

Removal of loose bodies is a common reason physicians perform hip surgery. Loose bodies may often be the result of trauma, such as a fall, an automobile accident, or a sports-related injury, or they may result from degenerative disease. When a torn labrum rubs continuously against cartilage in the joint, this may also cause fragments to break free and enter the joint. Loose bodies can cause a “catching” in the joint and cause both discomfort and pain. As with all arthroscopic procedures, the hip arthroscopy is undertaken with fluid in the joint, and there is a risk that some can escape into the surrounding tissues during surgery and cause local swelling. Moreover, the distention of the joint can result in a prolonged recovery time. Thus, there exists a need for improved systems and methods for performing minimally invasive procedures on the hip joint.

Laparoscopic and hysteroscopic methods have been used in the treatment of several conditions of the uterus including fibroids, polyps, and cancer. Typical endoscopes and hysteroscopes have diameters from 4-5 mm and may be inserted transcervically or through incisions in the abdomen or vagina. Endoscopes of smaller diameter are typically used for visual diagnostic purposes only as they cannot accommodate tools needed to perform procedures including resection and biopsy. However, patient comfort and recovery time are both negatively affected by large diameter tools, large incisions, and multiple procedures including separate diagnostic and surgical entries. Therefore, there is a need for endoscopes, laparoscopes and hysteroscopes having a small diameter that can simultaneously observe and treat uterine conditions.

### SUMMARY OF THE INVENTION

The present disclosure relates to systems and methods utilizing a small diameter imaging probe (e.g., endoscope) and a small diameter surgical tool for simultaneously imaging and performing a minimally invasive procedure on an internal structure within a body. More particularly, a small diameter imaging probe and a small diameter tool, such as an arthroscopic tool, can each include distal ends operatively configured for insertion into a narrow access space, for example, an access space less than 4 mm across at the narrowest region, more preferably less than 3 mm across at the narrowest region, and for many embodiments preferably less than 2 mm across at the narrowest region. Thus, for example, the imaging probe and arthroscopic tool of preferred embodiments of the invention are characterized by having a distal end with a diameter of less than 4 mm across at the largest cross-sectional region, more preferably less than 3 mm across at the largest region and most preferably less than 2 mm across at the largest region of each device.

In some embodiments, the body region may be accessed, for example, through a joint cavity characterized by a narrow access space. Example procedures which may require access via a joint cavity characterized by such a narrow access space may include procedures for repairing damage to the meniscus in the knee joint and procedures for repairing damage to the labrum in the hip and shoulder joints, for example. Advantageously, the systems and methods described herein enable accessing, visualizing and performing a procedure on a damaged region accessed via a joint cavity without the need for distension or other expansion of the joint cavity, for example, by injection of fluids under pressure or dislocation of the joint. Thus, the systems and methods of the present disclosure enable significant improvements in speeding up recovery time and preventing and/or mitigating complications. It will be appreciated that the arthroscopic tool of preferred embodiments can be used for performing a procedure on a damaged region that meets the dimensional requirements and that enables alignment with the visualization system described herein.

In exemplary embodiments, the imaging probe may enable visualization of both the target region and the arthroscopic tool thereby providing real-time visual feedback on a procedure being performed by the arthroscopic tool, for example a surgical procedure. It will be appreciated that the arthroscopic tool may be any arthroscopic tool such as for cutting, abrading or treating a body surface, or otherwise perform a procedure on a target region.

In some embodiments, the imaging probe may be characterized by an offset field of view, for example, offset from an insertion axis wherein the distal end of the imaging probe enables viewing at a non-zero angle relative to the insertion axis. In example embodiments, the field of view may include an offset axis having an angle relative to the insertion axis in a range of 5-45 degrees. Advantageously, the offset field of view may enable improved visualization of the target region and/or of the arthroscopic tool.

In some embodiments, the distal ends of the imaging probe and/or arthroscopic tool may be operatively configured for insertion into an access space having a predefined geometry, for example a curved geometry. Thus, for example, the distal ends of the imaging probe or endoscope and/or arthroscopic tool may include one or more regions shaped to substantially match a predefined geometry, for example, shaped to include a particular curvature to improve access to the region of interest. Example predefined geometries may include the curved space between the femoral head and the acetabulum in the hip joint or the curved space between the head of the humerus and the glenoid fossa of scapula in the shoulder joint. In some embodiments, the predefined geometry may be selected based on patient demographics, for example, based on age, gender, or build (i.e., height and weight).

In exemplary embodiments, the systems and methods may utilize one or more cannulas in conjunction with the imaging probe and/or arthroscopic tool described herein. In some embodiments, the cannula may be a single port cannula defining a single guide channel for receiving the imaging probe or arthroscopic tool therethrough. Alternatively, the cannula may be a dual port cannula, defining a pair of guide channels for receiving, respectively, the imaging probe and arthroscopic tool. In the dual port configuration, the cannula may be used to advantageously define a relative spatial positioning and/or orientation along one or more axes between the imaging probe and arthroscopic tool. For example, in some embodiments, the cannula may constrain the relative positioning of the imaging probe and arthroscopic tool to movement along each of the insertion axes defined by the guide channels, in yet further embodiments, the cannula may fix the orientation of the imaging probe and/or arthroscopic tool within its guide channel, for example to fix the orientation relative to the position of the other port. Thus, the cannula may advantageously be used to position and/or orientate the imaging probe and arthroscopic tool relative to one another, for example, in vivo, thereby enabling alignment of the field of view of the imaging probe with an operative portion or region of the body being treated with the arthroscopic tool.

Advantageously, a cannula as described herein may be operatively configured for insertion along an entry path between an entry point (for example, an incision) and an access space of a region of interest. In some embodiments, the cannula may be configured for insertion into the access space of the target region, for example, at least part of the way to the treatment site. Alternatively, the cannula may be configured for insertion along an entry path up until the access space with only the imaging probe and/or arthroscopic tool entering the access space. In some etribodiments, the cannula may be configured for insertion via an entry path having a predefined geometry and may therefore be shaped to substantially match the predefined geometry. In some embodiments, the predefined geometry of the entry path and the predefined geometry of the access space may be different. Thus, in exemplary embodiments, the cannula may be used to define a predefined geometry along the entry path up until the access space while the distal end(s) of the imaging probe and/or arthroscopic tool protruding from a distal end of the cannula may be used to define the predefined geometry along the access space. For example, the cannula may be used to define a relatively straight entry path up until the access space, and the distal ends of the imaging probe and/or arthroscopic tool may be used to define a curved path through the access space in some embodiments, the distal end(s) of the imaging probe and/or arthroscopic tool may include a resilient bias with respect to a predetermined geometry of the access space. Thus, the cannula may be used to rigidly constrain the shape of the distal end(s) up until the point of protrusion. Thus the positioning of the distal end of the cannula may, for example, determine a point at which the insertion path changes, for example from a straight entry path to a curved path through the access space.

In some embodiments, the cannula(s) or the visualization device or the arthroscopic tool may include a port for delivering medication or another therapeutic agent to the joint in question. For example, the arthroscopic tool may include an injection/delivery port for injecting/delivering a stem cell material into a joint cavity, and more particularly, with respect to a cartilage area of the target region, e.g., to facilitate repair thereof.

In accordance with the arthroscopic surgical method described herein, a patient was prepped and draped for a lateral menisectomy. No leg holder or post was employed to allow for limb flexibility. The patient was draped and sterile tech applied as is standard. No forced insufflation of the joint via pump or gravity flow was employed as would traditionally occur. The injection port was employed for any aspiration or delivery of saline required to clear the surgical field. Empty syringes were used to clear the view when occluded by either synovial fluid or injected saline. No tourniquet was employed in the case. A modified insertion port (from traditional arthroscopy ports) was chosen for insertion of the cannula and trocar. The position (lower) was modified given the overall size and angle aperture of the scope (1.4 mm gets around easily and 0 degree) that allows the user to migrate through the joint without distension. Following insertion of the endoscopic system and visual confirmation of the lateral meniscus tear, a surgical access port was established with the use of a simple blade. Under direct visualization and via the access port, traditional arthroscopic punches were employed (straight, left/right and up/down) to trim the meniscus. Visualization was aided during these periods by the injection of sterile saline 40 via a tubing extension set in short bursts of 2 to 4 cc at a time. Leg position via figure four and flexion/extension were employed throughout the procedure to open access and allow for optimal access to the site. Alternatively, a standard shaver hand piece was inserted into the surgical site to act as a suction wand to clear the site of any fluid or residual saline/synovial fluid. Multiple cycles of punches, irrigation and suctioning of the site were employed throughout the procedure to remove the offending meniscal tissue. Following final confirmation of correction and the absence of any loose bodies, the surgical site was sutured closed while the endoscope's side was bandaged via a band-aid. Preferably, both arthroscopic ports are closed without suturing due to the small size.

In a preferred embodiment, a wireless endoscopy system is configured to broadcast low-latency video that is received by a receiver and displayed on an electronic video display. The system operates at a video rate such that the user, such as a surgeon, can observe his or her movement of the distal end of the endoscope with minimal delay. This minimal configuration lacks the storage of patient data and procedure imagery, but compared to existing endoscopy systems it provides the benefits of a low number of components, low cost, and manufacturing simplicity. In a second embodiment, the wireless endoscopy system is configured to broadcast low-latency video to an electronic video display and also to a computer or tablet that executes application software that provides one or more of: patient data capture, procedure image and video storage, image enhancement, report generation, and other functions of medical endoscopy systems.

Preferred embodiments relate to a high-definition camera hand-piece that is connected to a control unit via a multi-protocol wireless link. In addition to the image sensor, the high definition camera unit contains a power source and associated circuitry, one or more wireless radios, a light source, a processing unit, control buttons, and other peripheral sensors. The control unit contains a system on chip (SOC) processing unit, a power supply, one or more wireless radios, a touchscreen enabled display, and a charging cradle for charging the camera hand-piece. By connecting the camera unit to the control unit in this way, this invention provides a real-time high definition imaging system that is far less cumbersome than traditional hard-wired systems.

In some embodiments, a cannula has ports for illumination, observation, and surgical tools to operate upon a patient. The cannula may be adapted for use in the uterine cavity and, more particularly, for the removal, resection, or excision of fibroids, polyps, or cancerous masses. The cannula can contain an endoscope and surgical tools such as forceps, cutting tools, abrading tools, mechanized rotary cutters, electrosurgical tools, lasers, snares, morcellators, RF cutting elements and electrically powered tools.

### BRIEF DESCRIPTION OF THE DRAWINGS

The foregoing and other objects, features and advantages of the invention will be apparent from the following more particular description of preferred embodiments of the invention as illustrated in the accompanying drawings in which like reference characters refer to the same parts throughout the different views. The drawings are not necessarily to scale, emphasis instead being placed upon illustrating the principles of the invention.

FIG. 1A illustrates a schematic illustration of a miniature endoscope system according to a preferred embodiment of the invention;

FIG. 1B illustrates components of an endoscope system in accordance with preferred embodiments of the invention;

FIG. 1C illustrates the assembled components of the embodiment of FIG. 1B;

FIG. 1D illustrates a side sectional view of the distal end of the sheath;

FIG. 1E illustrates a sectional view of the endoscope within the sheath;

FIG. 1F shows a sectional view of the proximal end of the sheath around the endoscope lens housing;

FIG. 2 is a cutaway view of a knee joint with cannulas inserted;

FIGS. 3A and 3B are cut away and sectional views of cannulas in a knee joint and the cannula for viewing;

FIG. 4 is a close-up view of the miniature endoscope and surgical cannula proximate to a surgical site;

FIG. 5A is a schematic view of the miniature endoscope with cannula system;

FIG. 5B shows a single cannula system with visualization and surgical devices inserted;

FIG. 5C shows a single cannula system with flexible tool entry;

FIGS. 5D and 5E show alternative parts for a single cannula two channel system;

FIG. 6 is a sectional view of the surgical system positioned relative to the meniscus;

FIG. 7A is a sectional view of the distal end of the cannula;

FIG. 7B is a sectional view of the distal end of the cannula taken along the line 7B of FIG. 7A;

FIG. 8 is a close-up view of the cannula adjacent a meniscus;

FIG. 9 illustrates a schematic illustration of a miniature endoscope system for facilitating a hip joint procedure, the system including an imaging probe assembly and a surgical tool assembly, according to a preferred embodiment of the invention;

FIGS. 10A-C depict sectional views of the endoscope system and hip joint of FIG. 9, illustrating various examples of distal end configurations of the imaging probe assembly and the surgical tool assembly of FIG. 9, according to preferred embodiments of the invention.

FIG. 11 depicts a schematic illustration of a miniature endoscope system for facilitating a hip joint procedure, the system including an imaging probe assembly and a surgical tool assembly sharing an integrally formed dual-port cannula, according to a preferred embodiment of the invention;

FIG. 12 depicts a section view of the endoscope system and hip joint of FIG. 11, illustrating an exemplary distal end configuration of the imaging probe assembly and the surgical tool assembly of FIG. 11, according to a preferred embodiment of the invention;

FIGS. 13A and 13B depict a function of surgical tool exhibiting a resilient bias with respect to a predefined curvature, according to a preferred embodiment of the invention; and

FIGS. 14 and 15 depict schematic and sectional illustrations of a miniature endoscope system for facilitating a shoulder joint procedure, the system including an imaging probe assembly and a surgical tool assembly, according to a preferred embodiment of the invention.

FIG. 16A illustrates an endoscope and sheath assembly with a distal prism lens system for angled viewing;

FIG. 16B illustrates a preferred embodiment of the invention in which the prism optical assembly is incorporated into the sheath;

FIG. 17 is a schematic diagram of the camera head and control system;

FIG. 18 illustrates the modular endoscope elements and data connections for a preferred embodiment of the invention.

FIG. 19A is a block diagram of the preferred embodiment of an endoscopy system pursuant to the present invention;

FIG. 19B is a block diagram of another embodiment of the endoscopy system pursuant to the present invention;

FIG. 19C is a block diagram of another embodiment of the endoscopy system pursuant to the present invention;

FIG. 20 is a perspective illustration of a camera handpiece of the endoscopy system;

FIG. 21A is a block diagram of an embodiment of elements of the endoscopy system;

FIG. 21B is a block diagram of an embodiments of additional elements of the endoscopy system;

FIG. 22 is a block diagram of RF energy, displays, and software associated with the endoscopy system;

FIG. 23 is a labelled block diagram of elements of the endoscopy system.

FIGS. 24A and 24B are diagrams showing the wireless endoscopy system with an HDMI formatted output from the camera module.

FIG. 25 is a diagram showing the wireless endoscopy system without an HDMI formatted output from the camera module, and the addition of an HDMI transmitter.

FIG. 26 illustrates components of a camera handpiece configured for a wired connection to a CCU.

FIGS. 27A and 27B illustrate devices to treat conditions of the uterus according to various embodiments.

FIG. 28 illustrates the distal tip of a cannula according to various embodiments.

FIGS. 29A-G illustrate various illumination systems of the present invention in accordance with preferred embodiments.

FIGS. 30A-30C illustrate embodiments of an endoscope having a sensor at the distal tip according to various embodiments.

### DETAILED DESCRIPTION OF THE INVENTION

Preferred embodiments of the invention are directed to devices and methods for minimally invasive diagnostic and/or surgical procedures. A first percutaneous entry position is used to insert a small diameter endoscope as described in greater detail below and/or in accordance with devices and methods described in U.S. Pat. No. 7,942,814 and U.S. application Ser. No. 12/439,116 filed on Aug. 30, 2007, and also in U.S. application Ser. No. 12/625,847 filed on Nov. 25, 2009, the entire contents of these patents and applications being incorporated herein by reference.

The present invention enables the performance of surgical procedures without the use of distension of the joint. Without the application of fluid under pressure to expand the volume accessible, a much smaller volume is available for surgical access. Existing techniques employ a pump pressure of 50-70 mmHg to achieve fluid distension of knee joints suitable for arthroscopic surgery. A tourniquet is used for an extended period to restrict blood flow to the knee. The present invention provides for the performance of arthroscopic procedures without fluid distension and without the use of a tourniquet. Low pressure flushing of the joint can be done using, for example, a manual syringe to remove particulate debris and fluid. In the course of certain procedures, distension of a joint may be used in conjunction with embodiments of the present invention. The present invention may also be used to image other internal tissues and cavities of a patient as described herein in greater detail.

A preferred embodiment of the invention utilizes positioning of the knee in a “figure four” position to achieve separation of the femur from the tibia to provide access. A preferred embodiment of the invention utilizes positioning of the knee in a “figure four” position to achieve separation of the femur from the tibia to provide access. This orientation provides a small aperture in which to insert devices into the joint cavity to visualize and surgically treat conditions previously inaccessible to larger-sized instruments.

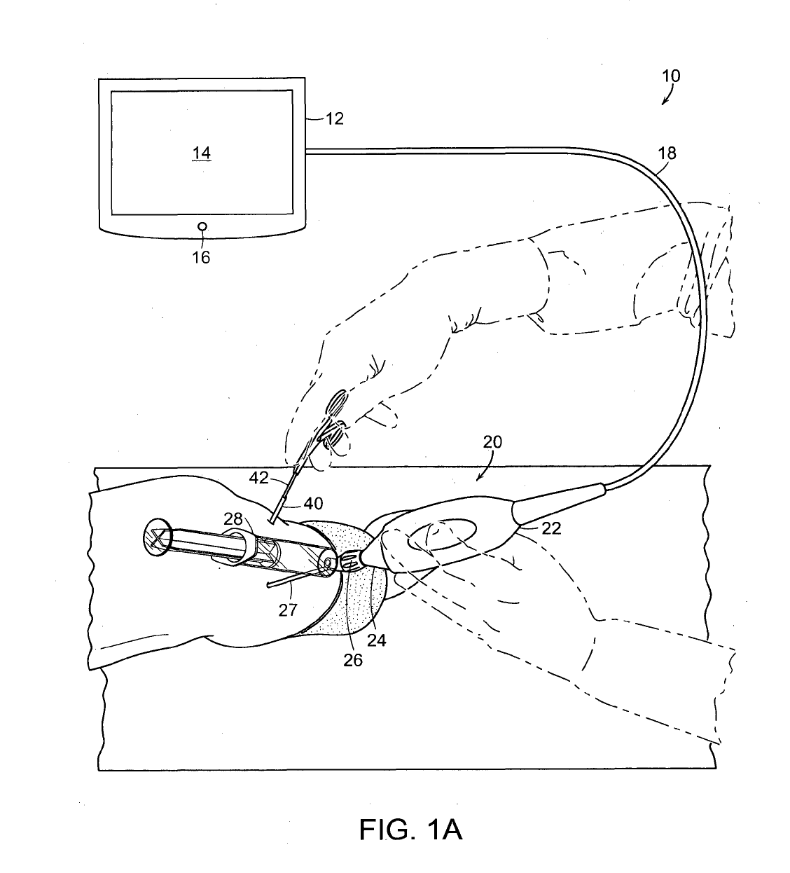

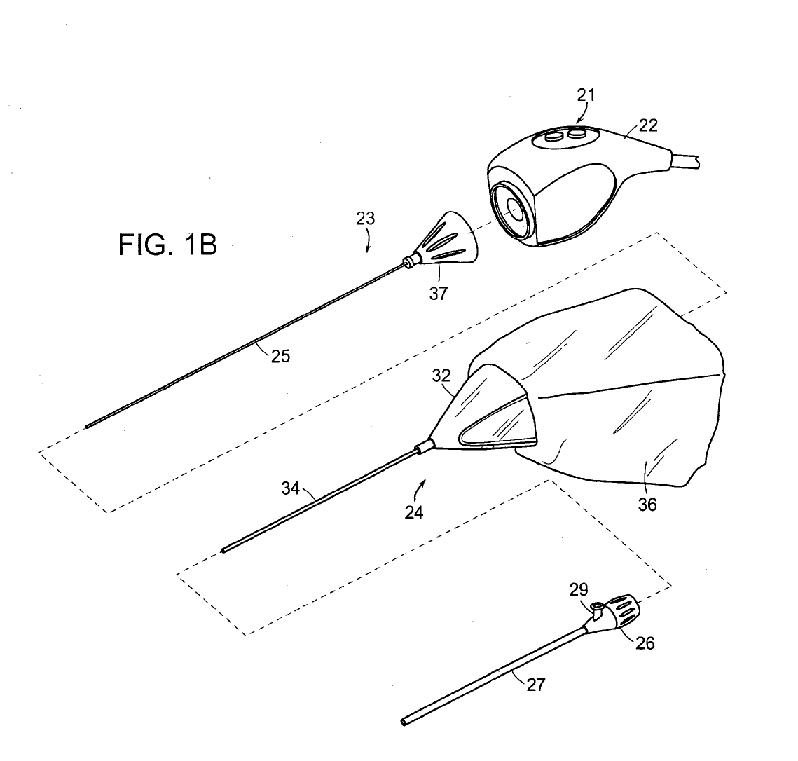

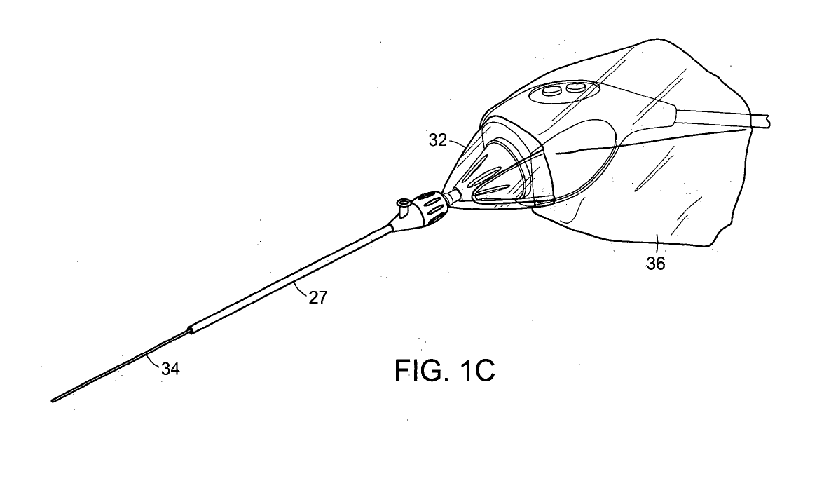

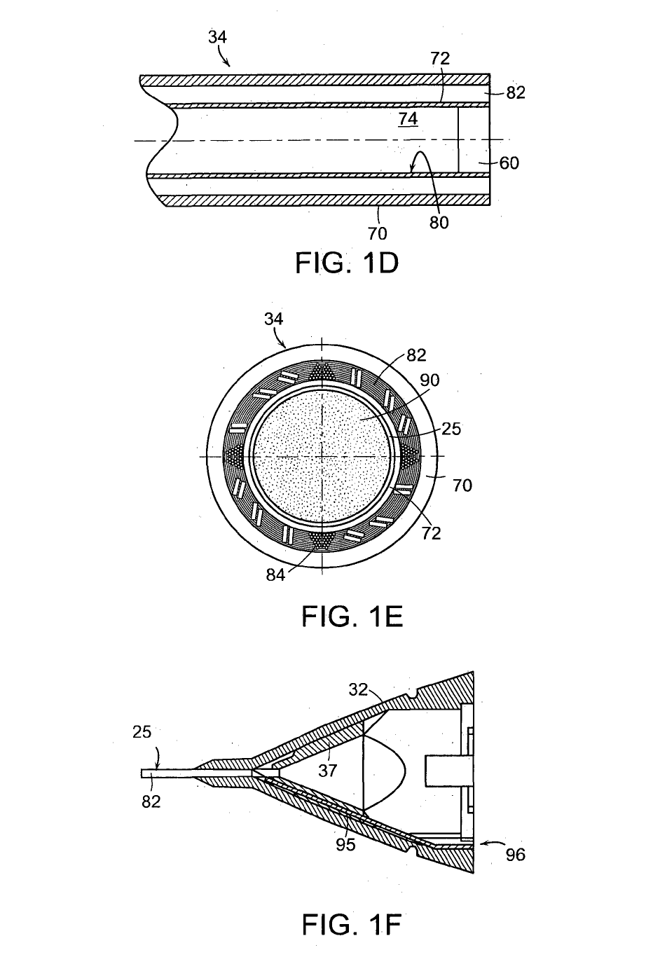

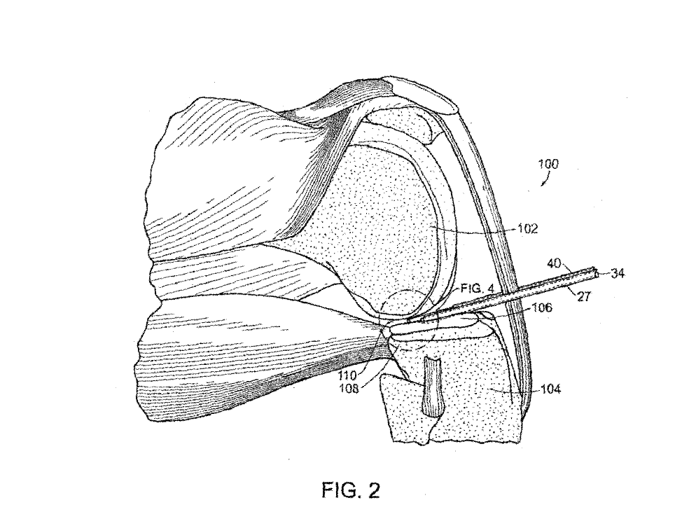

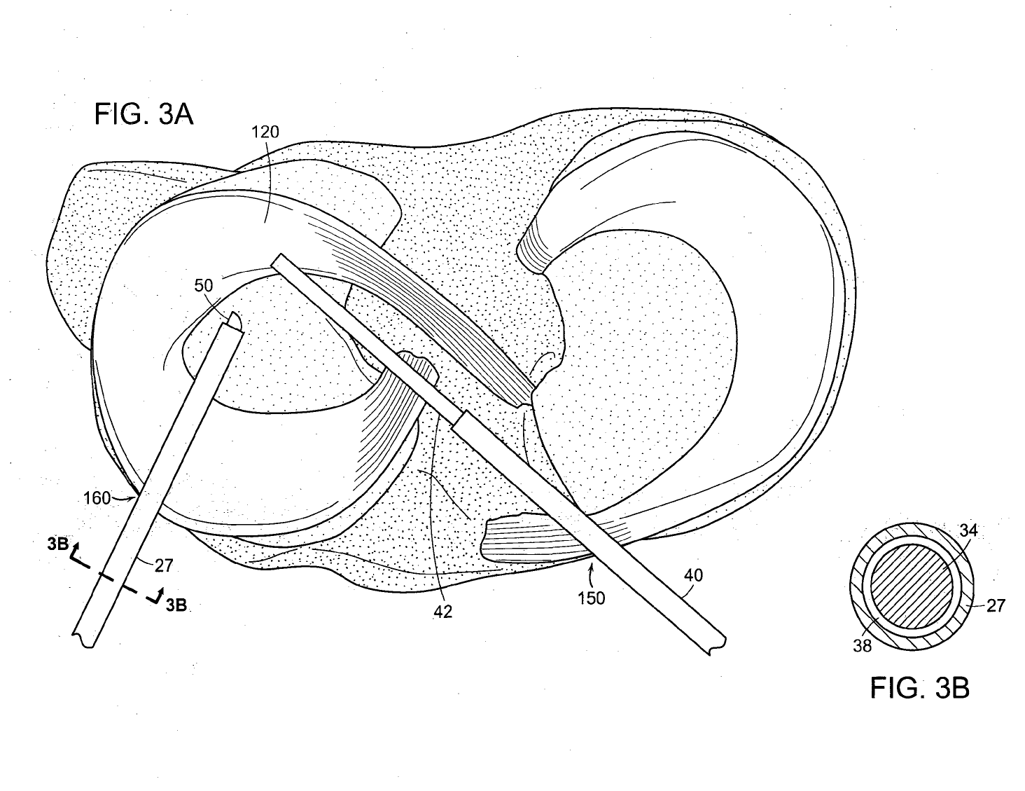

As depicted in FIG. 1A, a surgical system 10 includes an endoscope 20 attached to a handheld display device 12 having a touchscreen display 14 operated by one or more control elements 16 on the device housing 12. The system employs a graphical user interface that can be operated using touchscreen features including icons and gestures associated with different operative features of the endoscope. The display housing 12 can be connected to the endoscope handle 22 by a cable 18, or can be connected via wireless transmission and reception devices located in both the housing 12 and within the endoscope handle 22. The handle is attached to an endoscope, a sheath 24 that forms a sterile barrier to isolate the patient from the endoscope, and a cannula 27 is attached to the sheath at the connector 26. Connector 26 can include a part for coupling to a fluid source, such as a syringe 28, which can also be used to suction fluid and debris from the joint cavity.

The handle 22 is configured to operate with one or more imaging sensors that are optically coupled to a fiber optic imaging bundle that extends within an imaging tube 25 as depicted in FIG. 1B. The handle 22 can also provide an image output through the connection between the handle and the display and elective storage device 12. Alternatively, the handle can be connected to a laptop or desktop portable computer by wired or wireless connection. The device 12 can also be connected by wired or wireless connection to a private or public access network such as the internet.

The handle 22 is attachable to an endoscope 23 which comprises the tubular body 25 and a base 37. The housing 37 includes one or more lens elements to expand an image from the fiber optic imaging bundle within the tubular body 25. The base also attaches the endoscope 23 to the handle 22.

The handle 22 can include control elements 21 to operate the handle. A sheath 24 includes a tubular section 34, a base or optical barrier 32 that optically encloses the housing 37 and a sleeve or camera barrier 36 that unfolds from the proximal end of the base 32 to enclose the handle and a portion of the cable 18. The user can either slide their operating hand within the barrier to grasp and operate, or can grasp the handle with a gloved handle that is external to barrier 36.

During a procedure, the user first inserts the cannula 27 through the skin of the patient and into the joint cavity. The endoscope tube 25 is inserted into a lumen within the sheath tube 34 which is enclosed at the distal end by a window or lens. The sleeve is extended over the handle 22, and the sheath and endoscope are inserted into the cannula.

The assembled components are illustrated in FIG. 1C. The distal end of the sheath tube 34 is illustrated in the cross-sectional view of FIG. 1D wherein optical fibers 82 are located within a polymer sheath 70 that is attached to an inner metal tube 72. A transparent element or window 60 is attached to a distal end of the sheath 70, such as by adhesive, to an inner wall 80 of tube 72, for example, to form a fluid tight seal. A plurality of between 20 and 1500 optical fibers are enclosed between the outer tubular body 70 and the inner tube 72 in a plurality of several rows, preferably between 2 and 5 rows in a tightly spaced arrangement 84 with the fibers abutting each other to form an annular array.

FIG. 1F shows a sectional view of the endoscope housing 37 situated within sheath 32. In this embodiment, optical fibers 82 are collected into a bundle 95 which optically couples to the handle at interface 96.

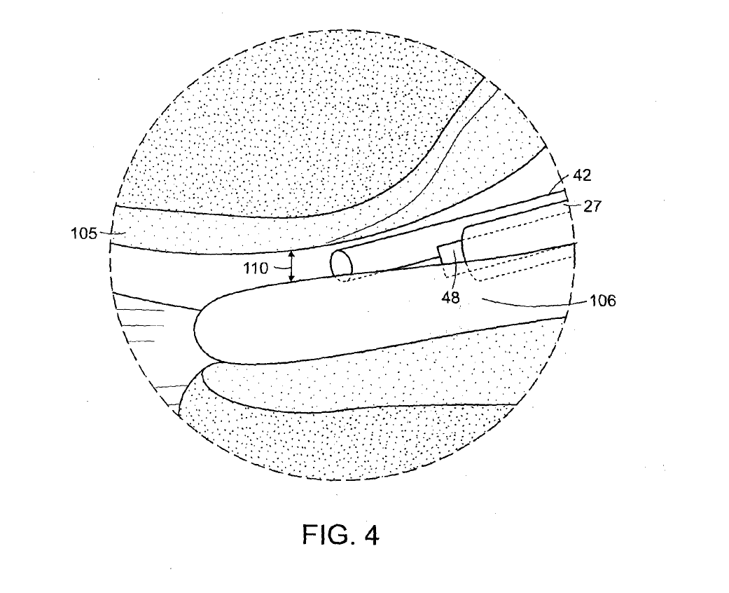

Shown in FIG. 2 is a side cut-away view of the procedure 100 being conducted that illustrates a meniscus repair procedure in which a surgical tool 42 is inserted into the cavity to reach the back of the meniscus 106 where injuries commonly occur. As no distension fluid is being used, the gap 110 between the articular cartilage covering the femur 102 and the meniscus is typically very small, generally under 4 mm in size and frequently under 3 mm in size. Thus, the distal end of the tool 42 that extends into the gap 110 is preferably under 4 mm in size, and generally under 3 mm to avoid damaging the cartilage and avoiding further damage to the meniscus. A cutting tool, an abrading tool, a snare, a mechanized rotary cutter, an electrosurgical tool or laser can be used to remove damaged tissue. The cannula 40 can also be used for precise delivery of tissue implants, medication, a stem cell therapeutic agent or an artificial implant for treatment of the site. This procedure can also be used in conjunction with treatments for arthritis and other chronic joint injuries.

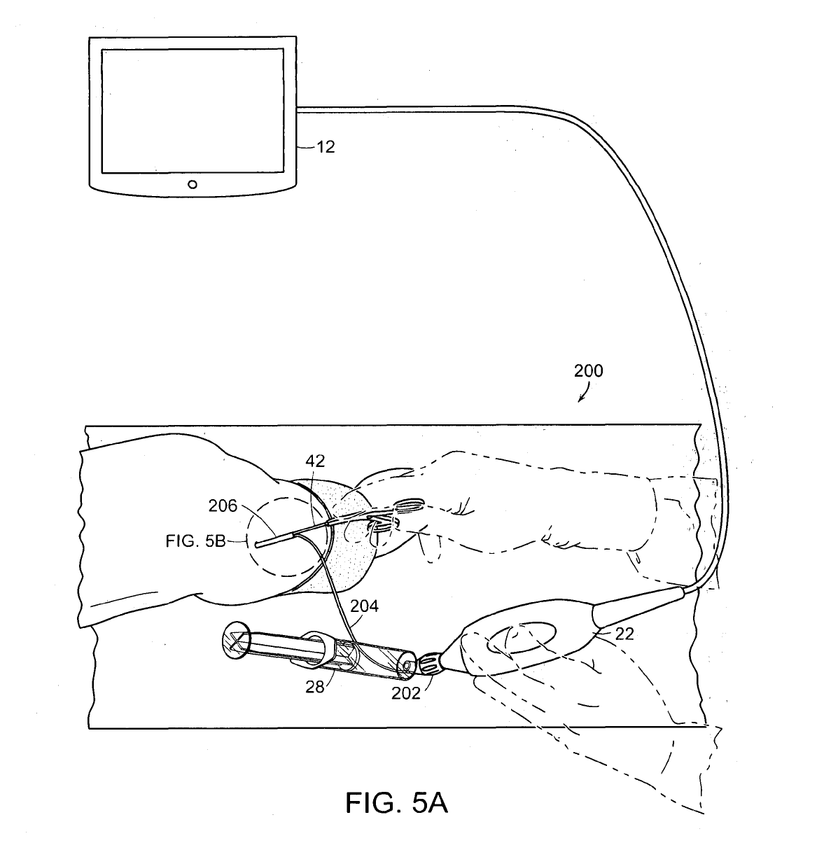

As seen in the view of FIG. 3A, the distal end of the tool is viewed with the miniature endoscope that is inserted through percutaneous entry point 160 with cannula 27. The tip of the sheath 50 can be forward looking along the axis of the cannula 27 or, alternatively, can have an angled lens system at the distal end of the endoscope that is enclosed with an angled window as shown. This alters the viewing angle to 15 degrees or 30 degrees, for example. Generally, the angle of view can be in a range of 5 degrees to 45 degrees in order to visualize the particular location of the injury under repair. As described previously in detail, the cross-sectional view of the cannula, sheath and endoscope system is depicted in FIG. 3B in which a gap 38 exists between the sheath 34 and the inner wall of the cannula to enable the transport of fluid and small debris.

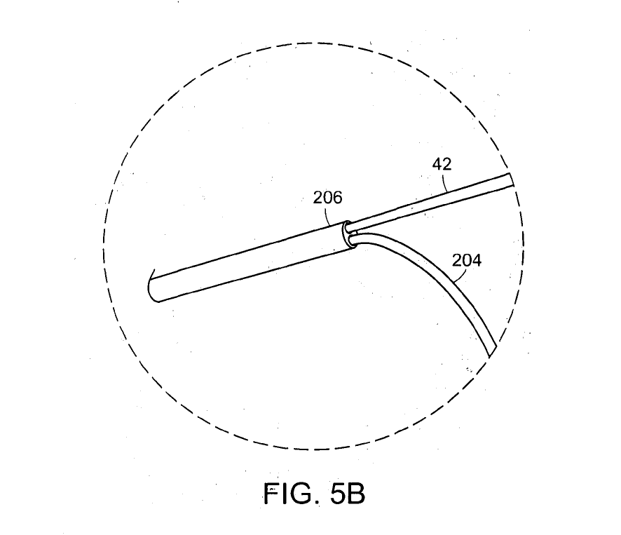

Shown in FIG. 4 is an enlarged view of region 108 in FIG. 2. As described, the gap 110 between the cartilage or overlying structures 105 and the surface of the meniscus 106 is very small such that proper placement of the tool 42 and the forward looking end of the sheath 48 through window 30 can only be achieved at diameters that are preferably under 3 mm.

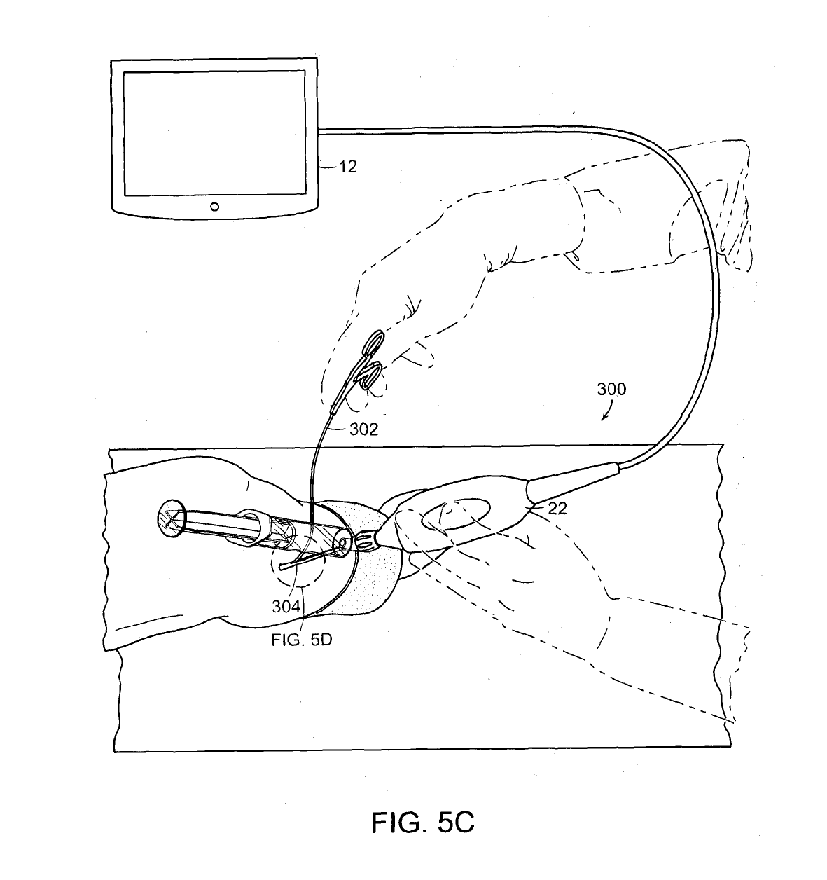

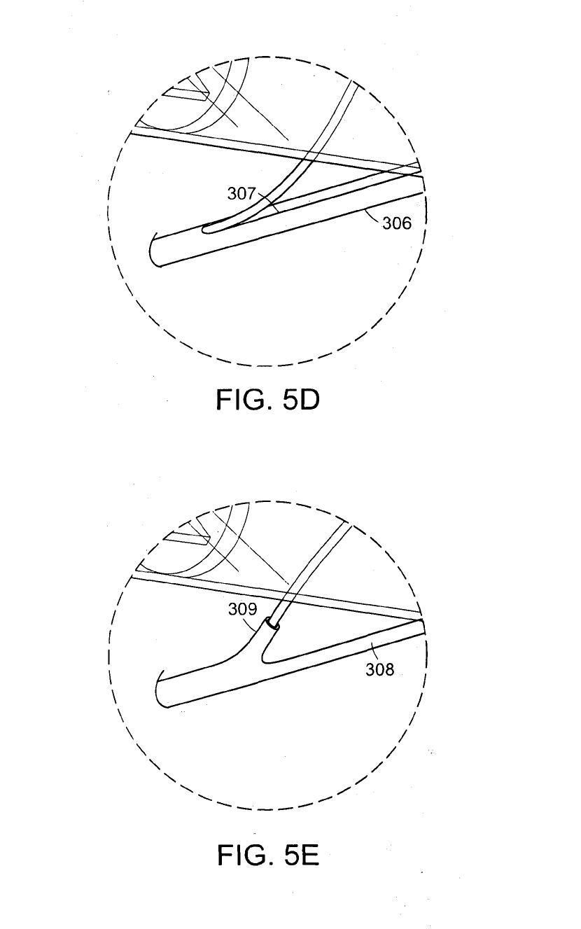

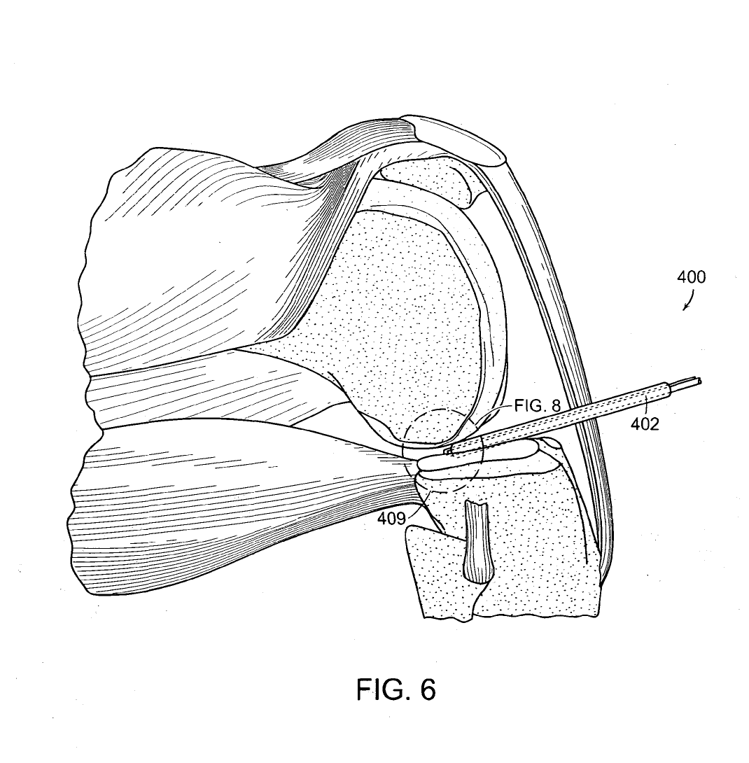

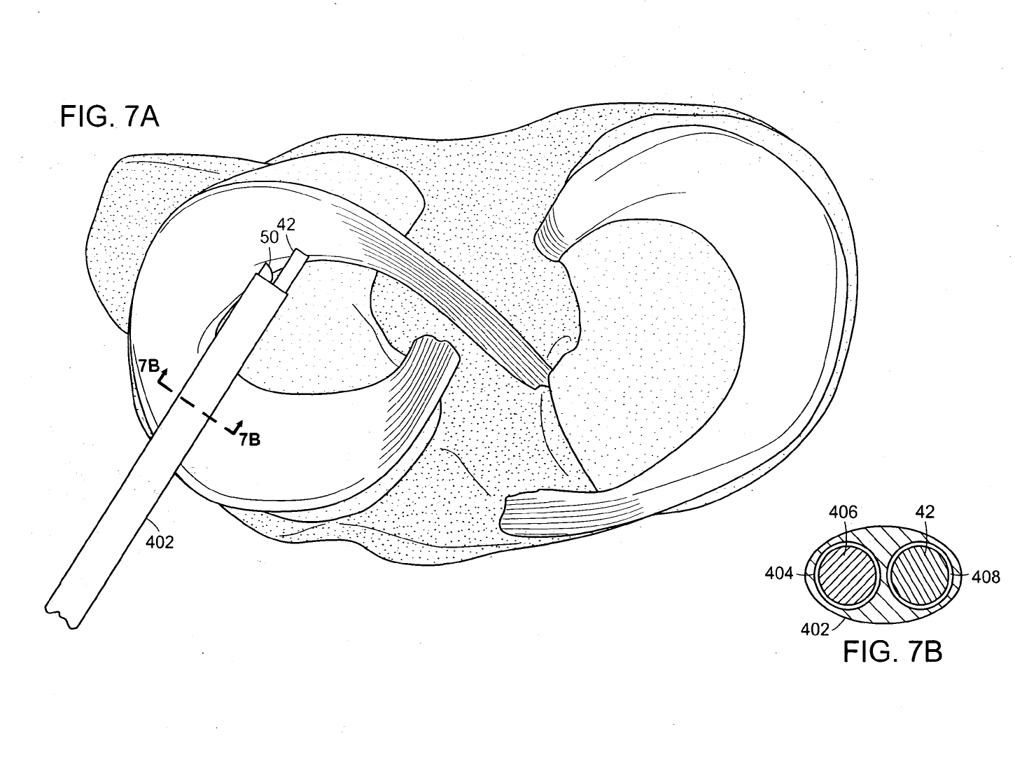

A single port system 200 for arthroscopic repair is shown in FIGS. 5A-8. As described before, a display is connected to endoscope handle 22; however, the sheath body can also include a port 202 to enable mounting of the syringe 28 to the sheath such that a fluid can be injected through a sheath channel.

A single cannula 206 can be used having a first channel to receive the flexible sheath and endoscope body. In this embodiment, the rigid tool 42 can be inserted straight through a second channel of the cannula 206. Note that the proximal end of the cannula 206 shown in FIG. 5B can be enlarged to provide for early manual insertion.

A further embodiment of a system 300 is shown in FIG. 5C wherein a single cannula 304 is used with a rigid sheath, as described previously, to be inserted through a first cannula channel, and a flexible tool shaft 302 is inserted through the second cannula channel. Note that both the tool and the sheath/endoscope combination can be flexible.

In the alternative embodiments illustrating cannula insertion, FIGS. 5D and 5E illustrate a side entry channel 307 for introduction of the flexible sheath or tool shaft on cannula 306 or a side port 309 for insertion of the flexible body and a straight shaft portion 308 for insertion of a rigid or flexible body.

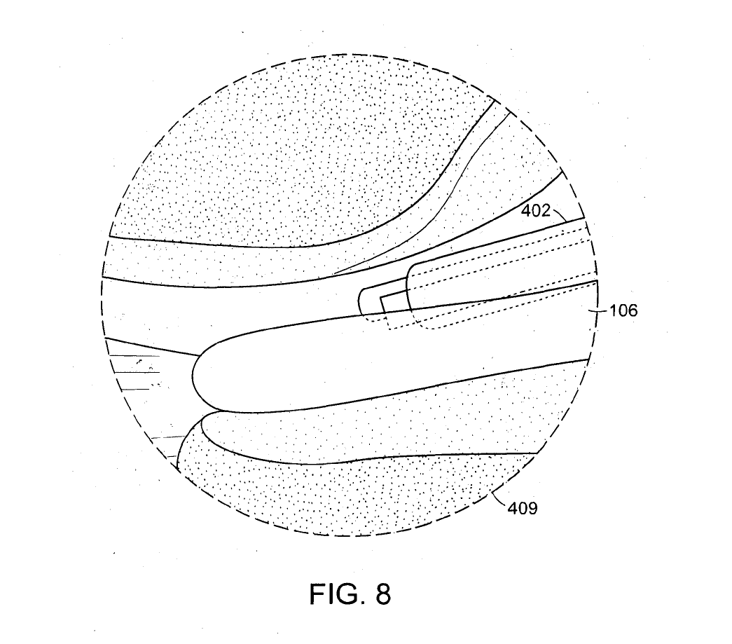

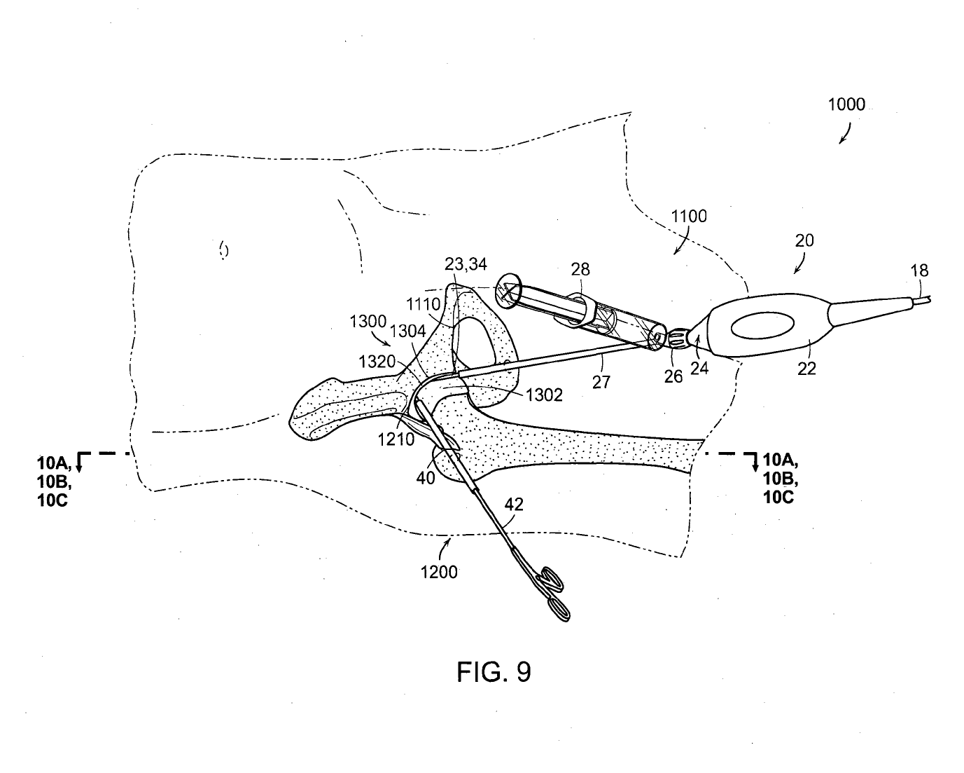

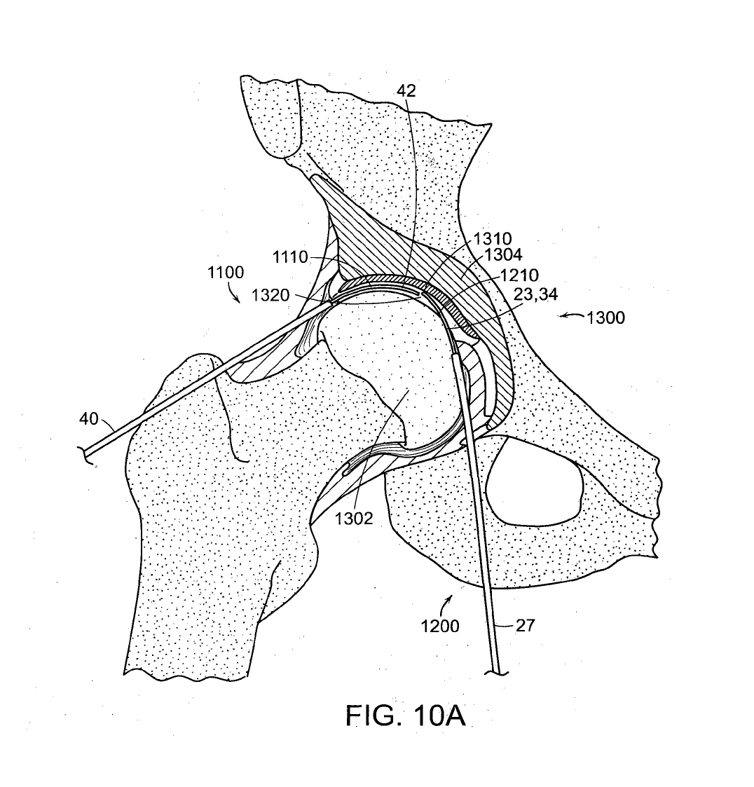

Shown in FIG. 6 is a cut-away view of a knee 400 in which a single port procedure is used with a cannula 402 having two channels as described herein. As seen in FIG. 7A, the cannula 402 has a first channel to receive the endoscope system in which a distal optical system 50 enables angled viewing of the distal end of the tool 42.

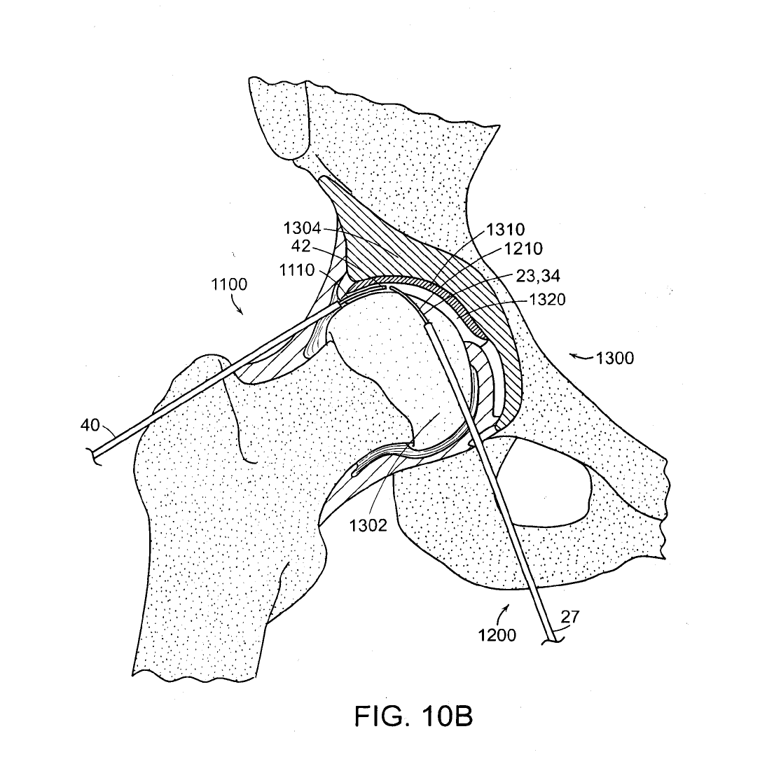

The cross-sectional view of the cannula 402 seen in FIG. 7B illustrates a first channel 404 for receiving the endoscope system 406 and a second channel 408 for receiving the tool 42. The cannula can include additional channels for fluid delivery and additional instruments or a separate suction channel. The cannula 402 can be rigid, semi-rigid or curved, depending on the application. The enlarged view of FIG. 8 illustrates the two-channel cannula inserted into the confined space of the knee joint, wherein the cannula can have a smaller diameter along one cross-sectional axis to enable insertion within the small joint cavity.

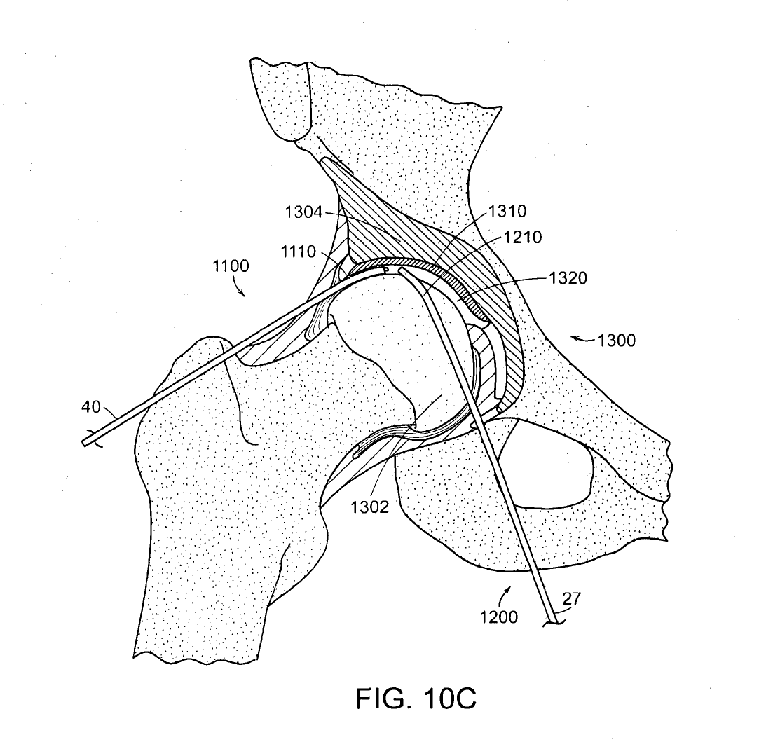

With reference to FIGS. 9, 10A, 10B, 10C, 11, 12, 13A and 13B, exemplary surgical systems and methods are illustrated for utilizing a small diameter imaging probe assembly 1100 and a small diameter surgical tool assembly 1200 for simultaneously imaging and performing a minimally invasive procedure on a damaged region 1310 of a hip joint 1300. In particular, the small diameter imaging probe assembly 1100 and small diameter surgical tool assembly 1200 may include distal ends (distal ends 1110 and 1210, respectively') operatively configured for insertion into a narrow access space 1320 defined by a cavity in hip joint 1300. For example, the distal ends 1110 and 1210 of the imaging probe assembly 1100 and surgical tool assembly 1200 may be operatively configured for insertion into an access space 1320 defined by a curved access space between the femoral head 1302 and the acetabulum 1304 in the hip joint 1300, such as the chondrolabral junction.

Advantageously, the distal ends 1110 and 1210 of the imaging probe assembly 1100 and surgical tool assembly 1200 may be dimensioned and shaped so as to enable access and visualization of the damaged region 1310 of the hip joint 1300 and performance of a surgical process on the damaged region 1310, all while minimizing the need for distension or other expansion of the joint cavity such as by injection of fluids and/or distraction of the hip joint 1300. Thus, the distal ends 1110 and 1210 of the imaging probe assembly 1100 and surgical tool assembly 1200 may be less than 4 mm in diameter, more preferably, less than 3 mm in diameter and most preferably less than 2 mm in diameter. Moreover, as depicted, the distal ends 11110 and 1210 of the imaging probe assembly 1100 and surgical tool assembly 1200 may be shaped to substantially match the curved access space 1320 between the femoral head 1302 and the acetabulum 1304 in the hip joint 1300. The various exemplary embodiments depicted in FIGS. 9, 10A, 10B, 10C, 11, 12, 13A and 13B are described in greater detail in the sections which follow.

With reference to FIG, 9, an exemplary surgical system 1000 is depicted. The exemplary surgical system 1000 includes a small diameter imaging probe assembly 1100 and a small diameter surgical tool assembly 1200 for simultaneously imaging and performing a minimally invasive procedure on a damaged region of a hip joint 1300.

As depicted, the imaging probe assembly 1100 may comprise an endoscopic system 20 similar to the endoscopic system 20 described with respect to FIGS. 1A-1F. Thus, for example, the endoscopic system 20, may be operatively associated with a display device, memory, processor, power source, and various other input and/or output devices (not depicted), for example, by way of cable 18 or a wireless connection. The endoscopic system 20 may include a handle 22 which may be configured to operate with one or more imaging sensors that are optically coupled to a fiber optic imaging bundle that extends within an imaging tube such as described above. The handle 22 can also provide an image output, for example, through the connection between the handle 22 and a display. In further embodiments, the handle 22 may be in operative communication with an external processing system such as a laptop, desktop portable computer, smartphone, PDA or other wireless mobile communication device. The endoscopic system 20 or associated architecture can also be connected by wired or wireless connection to a private or public access network such as the internet.

Similar to the setup in FIGS. 1A-F, the handle 22 of the endoscopic system 20 may be attachable to an endoscope 23, such as endoscope 23 of FIGS. 1A-F. The endoscopic system 20 may further include a sheath 34, such as sheath 34 of FIGS. 1A-F, configured for surrounding the endoscope 23, for example, for isolating the endoscope 23 from an external environment, and a cannula 27, such as cannula 27 of FIGS. 1A-F, configured for defining a guide channel for receiving the sheath 34 and endoscope 23 therethrough. The cannula 27 may further be associated with a connector 26 for connecting the cannula 27 relative to a base of the sheath 34 and for enabling fluid injection via a space between the cannula 27 and the sheath 34, for example, using injector 28.

With reference still to FIG. 9, the surgical tool assembly 1200 may include a surgical tool 42 and a cannula 40, for example, similar to the surgical tool 42 and cannula 40 described above with respect to FIGS. 1A-F. Commonly used surgical tools which may be used include, for example, a hook probe, used to assess the integrity and consistency of the hip, radiofrequency probes that ablate soft tissue and can also smoothen tissue surfaces, and various shavers or burrs that can take away diseased tissue. If the acetabular labrum requires repair, specially designed anchors may be also used. This is, however, by no means a comprehensive list of the surgical tools which may be used in conjunction with the systems and methods described herein.

In an exemplary arthroscopic hip procedure, the cannula 27 and 40 for the endoscopic system 27 and a surgical tool 42, may be inserted into a patient along entry paths defined between an entry point (for example, an incision) and an access space of a damaged region of the hip joint, for example, the curved access space 1320 between the femoral head 1302 and the acetabulum 1304 in the hip joint 1300, such as the chondrolabral junction. In some embodiments (see, e.g., FIG, 10C) the cannula 27 and 30 may be configured for insertion all the way into the curved access space 1320, for example, at least part of the way to the damaged region 1310 of the hip. In other embodiments (see, e.g., FIGS, 10A and 10B), the cannulas 27 and 40 may be configured for insertion along entry paths up until the start of the curved access space 1320. Thus, for example, the sheath/endoscope 23, 34 may extend/protrude from a distal end of the cannula 27 and/or the tool 42 may extend/protrude from a distal end of the cannula 40 in the curved access space 1320. In yet further exemplary embodiments, the cannula 27 and 40 may be configured for insertion via entry paths having predefined geometry. Thus the cannulas 27 and 40 may be shaped to substantially match the predefined geometry. It is noted that the systems and methods of the present disclosure are not limited to the depicted entry points and entry paths. Indeed, one of ordinary skill in the art would appreciate orthopedic surgeons typically have their own preferential configuration of entry points and entry paths for achieving access to the hip joint.

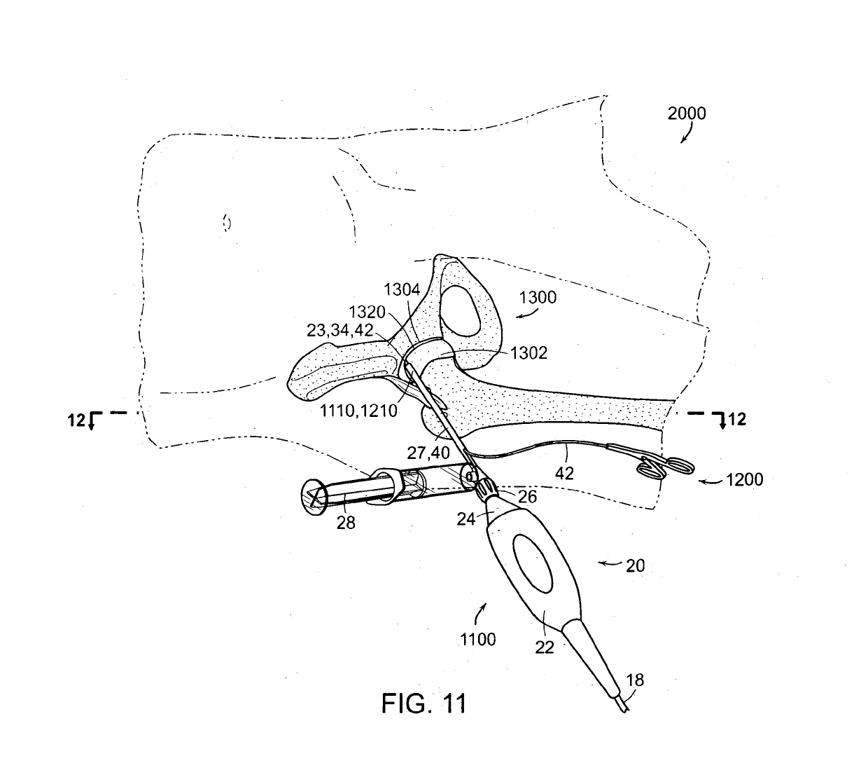

With reference now to FIG. 10A, a first embodiment of the distal ends 1110 and 1210 of the imaging probe assembly 1100 and surgical tool assembly 1200 of FIG. 9 is depicted taken along section 10A-10A of FIG. 9. As depicted, the cannulas 27 and 40 are inserted up until the start of the curved access space 1320 between the femoral head 1302 and the acetabulum 1304 in the hip joint 1300. The sheath/endoscope 34 and the tool 42 extend/protrude from distal ends of the cannula 27 and 40 into the curved access space 1320 to reach the damaged region 1310 of the hip joint 1300. As depicted, the distal ends 1110 and 1210 of the imaging probe assembly 1100 and surgical tool assembly 1200 are shaped to substantially match the curved access space 1320. Thus, in the depicted embodiment, distal ends of the tool 42 and of the sheath/endoscope 23, 34 are curved to substantially match the curvature of the curved access space 1320.

With reference now to FIG. 1013, a second embodiment of the distal ends 1110 and 1210 of the imaging probe assembly 1100 and surgical tool assembly 1200 of FIG. 9 is depicted taken along section 10B-10B of FIG. 9. Similar to the embodiment in FIG. 10A, the cannula 27 and 40 are depicted as inserted up until the start of the curved access space 1320 between the femoral head 1302 and the acetabulum 1304 in the hip joint 1300. Thus, the sheath/endoscope 34 and the tool 42 extend/protrude from distal ends of the cannula 27 and 40 into the curved access space 1320 to reach the damaged region 1310 of the hip joint 1300. As depicted, the distal ends 1110 and 1210 of the imaging probe assembly 1100 and surgical tool assembly 1200 are shaped to substantially match the curved access space 1320. Thus, in the depicted embodiment, distal ends of the tool 42 and of the sheath/endoscope 23, 34 are curved to substantially match the curvature of the curved access space 1320. In comparison with the embodiment of FIG. 10A, the depicted arch length and curvature of the distal ends of the tool 42 and of the sheath/endoscope 23, 34 in FIG. 10B are less than the depicted arch length and curvature of the distal ends of the tool 42 and of the sheath/endoscope 23, 34 in FIG. 10A. It will be appreciated by one of ordinary skill in the art that various geometric configurations may be utilized for accessing the curved access space 1320, for example, dependent on patient demographics such as age, build (e,g., height and weight), gender, patient physiology, damage region location, and other factors.

With reference now to FIG. 10C, a third embodiment of the distal ends 1110 and 1210 of the imaging probe assembly 1100 and surgical tool assembly 1200 of FIG. 9 is depicted taken along section 10C-10C of FIG. 9. In contrast with FIGS. 10A and 10B, the cannula 27 and 40 are depicted as inserted into the curved access space 1320 between the femoral head 1302 and the acetabulum 1304 in the hip joint 1300. Thus, the sheath/endoscope 34 and the tool 42 are substantially enclosed up to the damaged region 1310 of the hip joint 1300. As depicted, the distal ends 1110 and 1210 of the imaging probe assembly 1100 and surgical tool assembly 1200 are shaped to substantially match the curved access space 1320. Thus, in the depicted embodiment, distal ends of the cannula 27 and 40 are curved to substantially match the curvature of the curved access space 1320. Again, it will be appreciated by one of ordinary skill in the art that various geometric configurations may be utilized for accessing the curved access space 1320, for example, dependent on patient demographics such as age, build (e.g., height and weight), gender, patient physiology, damage region location, and other factors.

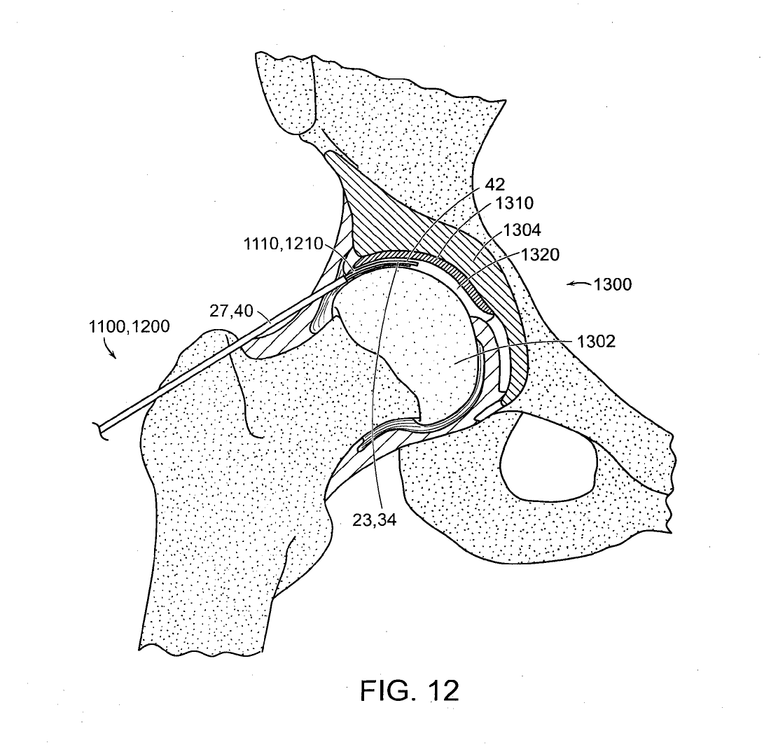

With reference now to FIG. 11, an further exemplary surgical system 2000 is depicted. The exemplary surgical system 2000, includes a small diameter imaging probe assembly 1100 and a small diameter surgical tool assembly 1200 for simultaneously imaging and performing a minimally invasive procedure on a damaged region of a hip joint 1300. As depicted, the imaging probe assembly 1100 may comprise an endoscopic system 20 similar to the endoscopic system 20 described with respect to FIGS. 1A-1F. Thus, for example, the endoscopic system 20, may be operatively associated with a display device, memory, processor, power source, and various other input and/or output devices (not depicted), for example, by way of cable 18 or a wireless connection. The endoscopic system 20 may include a handle 22 which may be configured to operate with one or more imaging sensors that are optically coupled to a fiber optic imaging bundle that extends within an imaging tube such as described above. The handle 22 can also provide an image output, for example, through the connection between the handle 22 and a display. In further embodiments, the handle 22 may be in operative communication with an external processing system such as a laptop, desktop portable computer, smartphone, PDA or other mobile device. The endoscopic system 20 or associated architecture can also be connected by wired or wireless connection to a private or public access network such as the internet.

Similar to the setup in FIGS. 1A-F, the handle 22 of the endoscopic system 20 may be attachable to an endoscope 23, such as endoscope 23 of FIGS. 1A-F. The endoscopic system 20 may further include a sheath 34, such as sheath 34 of FIGS. 1A-F, configured for surrounding the endoscope 23, for example, for isolating the endoscope 23 from an external environment, and a cannula 27, such as cannula 27 of FIGS. 1A-F, configured for defining a guide channel for receiving the sheath 34 and endoscope 23 therethrough. The cannula 27 may further be associated with a connector 26 for connecting the cannula 27 relative to a base of the sheath 34 and for enabling fluid injection via a space between the cannula 27 and the sheath 34, for example, using injector 28.

With reference still to FIG. 11, the surgical tool assembly 1200 may include a surgical tool 42 and a cannula 40, for example, similar to the surgical tool 42 and cannula 40 described above with respect to FIGS. 1A-F. Commonly used surgical tools which may be used include, for example, a hook probe, used to assess the integrity and consistency of the hip, radiofrequency probes that ablate soft tissue and can also smoothen tissue surfaces, and various shavers or burrs that can take away diseased tissue. If the acetabular labrum requires repair, specially designed anchors may be also used. This is, however, by no means a comprehensive list of the surgical tools which may be used in conjunction with the systems and methods described herein.

In contrast with the embodiment of FIG. 9, the embodiment in FIG. 11 depicts a dual port cannula, e.g., wherein the cannula 27 and cannula 40 are integrally formed as a single cannula defining a pair of guide channels for receiving, respectively, the sheath/endoscope 23, 34 and the surgical tool 42. In the dual port configuration, the integrally formed cannula 27 and 40 may be used to advantageously define a relative spatial positioning and/or orientation along one or more axes between the sheath/endoscope 23, 34 and the surgical tool 42. For example, the integrally formed cannula 27 and 40 may constrain the relative positioning of sheath/endoscope 23, 34 and the surgical tool 42 to movement along each of the insertion axes defined by the guide channels. In some embodiments, the integrally formed cannula 27 and 40 may also fix the orientation of the sheath/endoscope 23, 34 and/or the surgical tool 42 within its respective guide channel, for example to fix the orientation relative to the position of the other port. Thus, the integrally formed cannula 27 and 40 may advantageously be used to position and/or orientate the sheath/endoscope 23, 34 and/or the surgical tool 42 relative to one another, in vivo, thereby enabling alignment of the field of view of the imaging probe with an operative portion or target of the arthroscopic tool. 1t is noted that the embodiment of FIG. 11 is somewhat similar to the integrally formed dual port cannula embodiment described with respect to FIGS. 5A-E and the imaging probe assembly 1100 may employ, for example, an angularly offset viewing angle, for example, relative to the insertion access.

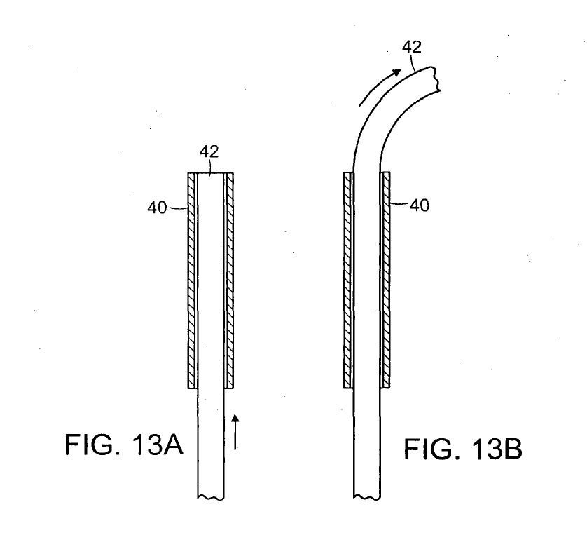

With reference now to FIG. 12, an example embodiment of the distal ends 1110 and 1210 of the imaging probe assembly 1100 and surgical tool assembly 1200 of FIG. 11 is depicted taken along section 12-12 of FIG. 11. As depicted the integrally formed cannula 27 and 40 is depicted as inserted up until the start of the curved access space 1320 between the femoral head 1302 and the acetabulum 1304 in the hip joint 1300. Thus, the sheath/endoscope 34 and the tool 42 extend/protrude from distal ends of the integrally formed cannula 27 and 40 into the curved access space 1320 to reach the damaged region 1310 of the hip joint 1300. As depicted, the distal ends 1110 and 1210 of the imaging probe assembly 1100 and surgical tool assembly 1200 are shaped to substantially match the curved access space 1320. Thus, in the depicted embodiment, distal ends of the tool 42 and of the sheath/endoscope 23, 34 are curved to substantially match the curvature of the curved access space 1320. It will be appreciated, however, that in some embodiments, the integrally formed cannula 27 and 40 may be inserted into the curved access space 1320 between the femoral head 1302 and the acetabulum 1304 in the hip joint 1300. Thus, in some embodiments, the distal ends of the integrally formed cannula 27 and 40 may be curved to substantially match the curvature of the curved access space 1320 (see, e.g., FIG. 10C). It will also be appreciated by one of ordinary skill in the art that various geometric configurations may be utilized for accessing the curved access space 1320, for example, depending on patient demographics such as age, build (e.g., height and weight), gender, patient physiology, damage region location, and other factors.

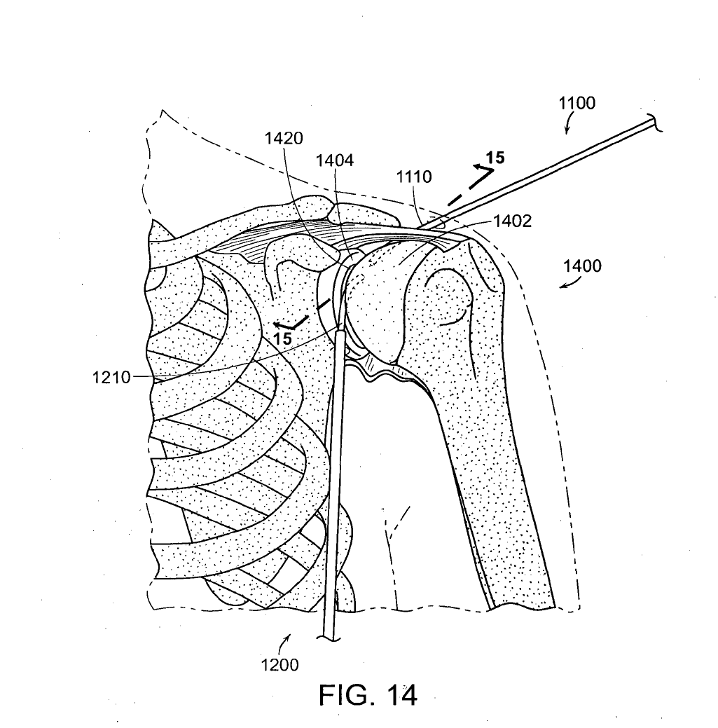

In some embodiments, the distal end(s) of the imaging probe and/or surgical tool may include a resilient bias with respect to a predetermined geometry of the access space. Thus, the imaging probe and/or surgical tool may advantageously bend in a predetermined manner upon protrusion from a cannula, e.g., to facilitate insertion into a curved access space. In such embodiments, the cannula may be used to rigidly constrain the shape of the distal end until the point of protrusion. Thus the positioning of the distal end of the cannula may, for example, determine a point at which the insertion path changes, for example from a straight entry path to a curved path through the curved access space. With reference to FIGS. 13A and 13B, an exemplary embodiment is depicted whereby a surgical tool 42 is configured to bend in a predetermined manner upon protrusion from the cannula 40.

It will be appreciated by one of ordinary skill in the art that any number of mechanisms may be used to cause a bend in a distal end of an imaging probe, surgical tool and/or cannula. For example, shape memory material (for example, heat sensitive shape memory materials), articulating segments, and other mechanisms may be utilized. In some embodiments, a cannula may include one or more telescopic distal portions. In exemplary embodiments, such telescopic distal portions may exhibit a resilient bias with respect to a predetermined geometry of the access space. In other embodiments, a cannula may include articulating segments which may be used to shape and steer the path of the cannula.

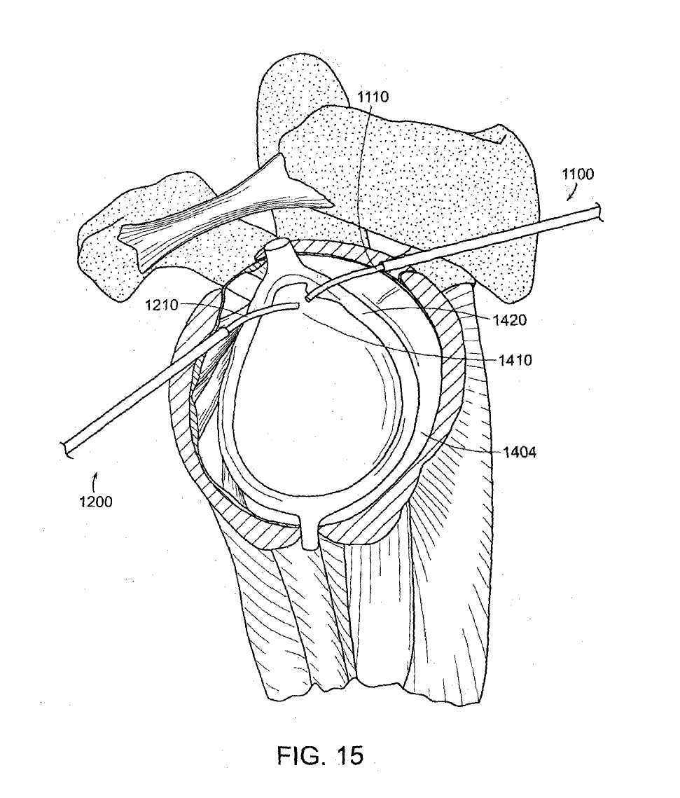

With reference now to FIGS. 14 and 15, exemplary surgical systems and methods are illustrated for utilizing a small diameter imaging probe assembly 1100 and a small diameter surgical tool assembly 1200 for simultaneously imaging and performing a minimally invasive procedure on a damaged region 1410 of a shoulder joint 1400. In particular, the small diameter imaging probe assembly 1100 and small diameter surgical tool assembly 1200 may include distal ends (distal ends 1110 and 1210, respectively) operatively configured for insertion into a narrow access space 1420 defined by a cavity in shoulder joint 1400. For example, the distal ends 1110 and 1210 of the imaging probe assembly 1100 and surgical tool assembly 1200 may be operatively configured for insertion into a curved access space 1420 defined between the head of the humerus 1402 and the glenoid fossa 1404 of scapula in the shoulder joint.

Advantageously, the distal ends 1110 and 1210 of the imaging probe assembly 1100 and surgical tool assembly 1200 may be dimensioned and shaped so as to enable access and visualization of a damaged region 1410 of the shoulder joint 1400 and performance of a surgical process on the damaged region 1410, all while minimizing the need for distension or other expansion of the joint cavity such as by injection of fluids and/or distraction of shoulder joint 1400. Thus, the distal ends 1110 and 1210 of the imaging probe assembly 1100 and surgical tool assembly 1200 may be less than 4 mm in diameter, more preferably less than 3 mm in diameter and most preferably less than 2 mm in diameter. Moreover, as depicted, the distal ends 1110 and 1210 of the imaging probe assembly 1100 and surgical tool assembly 1200 may be shaped to substantially match the curved access space 1420 between the head of the humerus 1402 and the glenoid fossa 1404 of scapula in the shoulder joint.

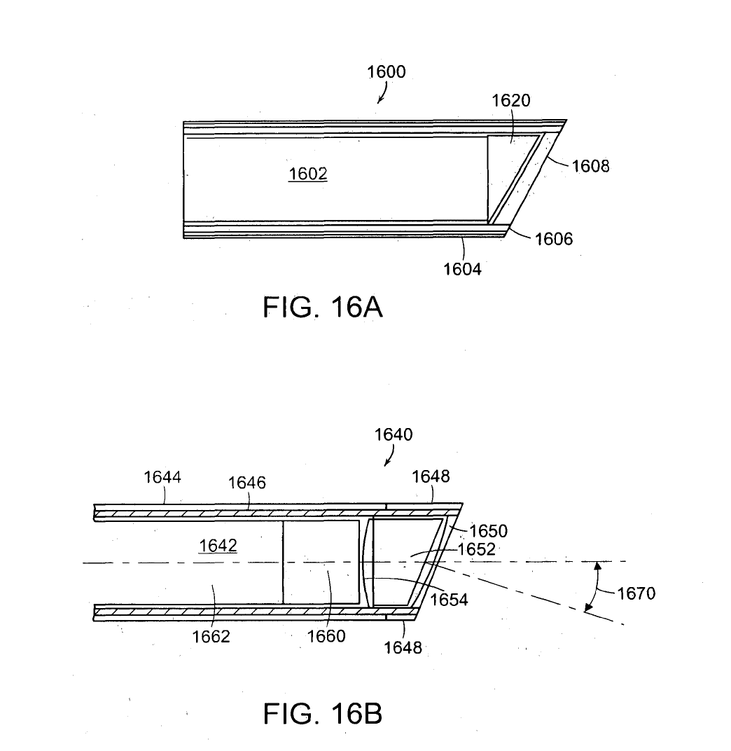

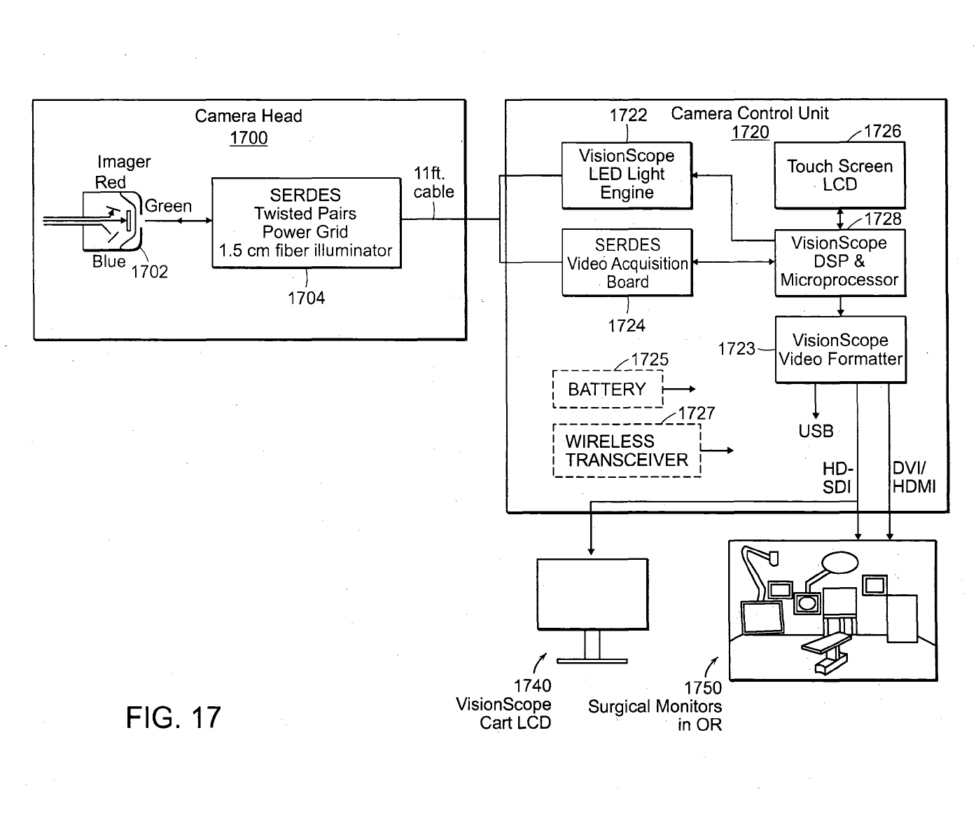

FIG. 16A illustrates a distal end of a sheath and endoscope assembly 1600 for angled viewing in which a distal prism lens system 1620 abuts an angled window 1608 that is sealed within the sheath tube 1604. Illumination fibers 1606 form an annular illumination ring to illuminate the field of view. The endoscope tube includes a fiber optic imaging bundle with a lens doublet positioned between the image bundle and prism 1620.

Shown in FIG. 16B is an endoscope and sheath assembly 1640 in which an endoscope 1642 as described herein comprises a fiber optic imaging bundle 1662 coupled to distal optics assembly 1660. The endoscope body 1642 slides into the sheath such that the distal optics assembly 1660 receives light from the sheath imaging optics, which can include a prism 1652, a proximal lens 1654 and a distal window 1650 having a curved proximal surface such that the endoscope views at an angle different from the endoscope axis, preferably at an angle between 5 degrees and 45 degrees, such as 30 degrees. The sheath can include a tube 1646 having an inner surface wherein an adhesive can be used to attach the peripheral surfaces of the prism 1652 and window 1650. In this embodiment the sheath imaging optics are matched to the endoscope optics to provide for an angled view of 30 degrees, for example.

The illumination fiber bundle 1644 can comprise an annular array of optical fibers within a polymer or plastic matrix that is attached to the outer surface of tube 1646. At the distal end of the illumination fiber assembly 1644 is a light transmitting sleeve 1648 that is shaped to direct light emitted from the distal ends of the fiber assembly 1644 at the correct angle of view. The sleeve 1648 operates to shape the light to uniformly illuminate the field of view at the selected angle. Thus, the sleeve's illumination distribution pattern will vary as a function of the angle of view 1670.

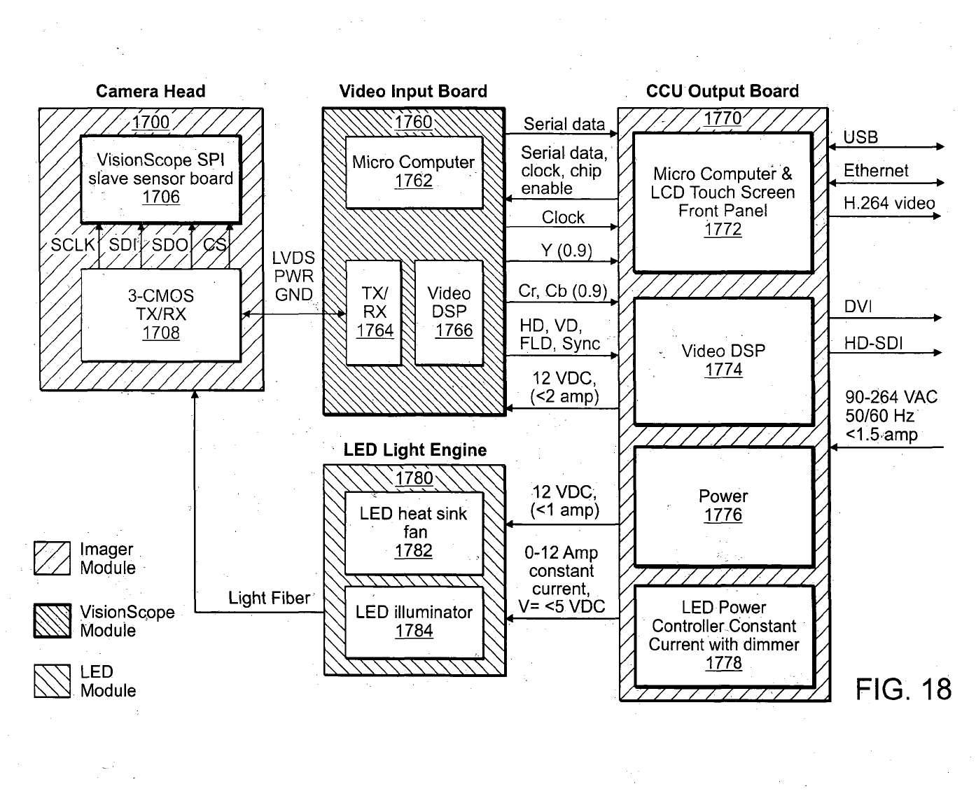

Illustrated in FIG. 17 are camera head 1700 and camera control unit 1720 features in accordance with the invention. The imager unit 1702 in the camera head 1700 may divide the incoming visual signals into red, green, and blue channels. The imager unit 1702 is in communication with the imager control unit 1704 to receive operating power. In addition, the imager control unit 1704 delivers illumination light to the endoscope while receiving imagery from the imager unit 1702. The camera control unit 1720 is connected by cable to the camera head 1700. LED illumination is delivered to the imager control unit 1704 from the LED light engine 1722. Imagery acquired by the endoscope system is delivered from the imager control unit 1704 to the video acquisition board 1724. The LED light engine 1722 and video acquisition board 1724 are in communication with the DSP and microprocessor 1728. The DSP and microprocessor 1728 is also equipped to receive input from a user of the system through the touchscreen LCD 1726. The DSP and microprocessor conducts data processing operations on the clinical imagery acquired by the endoscope and outputs that data to the video formatter 1723. The video formatter 1723 can output video in a variety of formats including as HD-SDI and DVI/HDMI, or the video formatter can simply export the data via USB. An HDI-SDI or DVI/HDMI video signal may be viewed on standard surgical monitors in the OR 1750 or using a LCD display 1740. The handle can include a battery 1725 and a wireless transceiver 1727 to enable a cableless connection to a base unit.

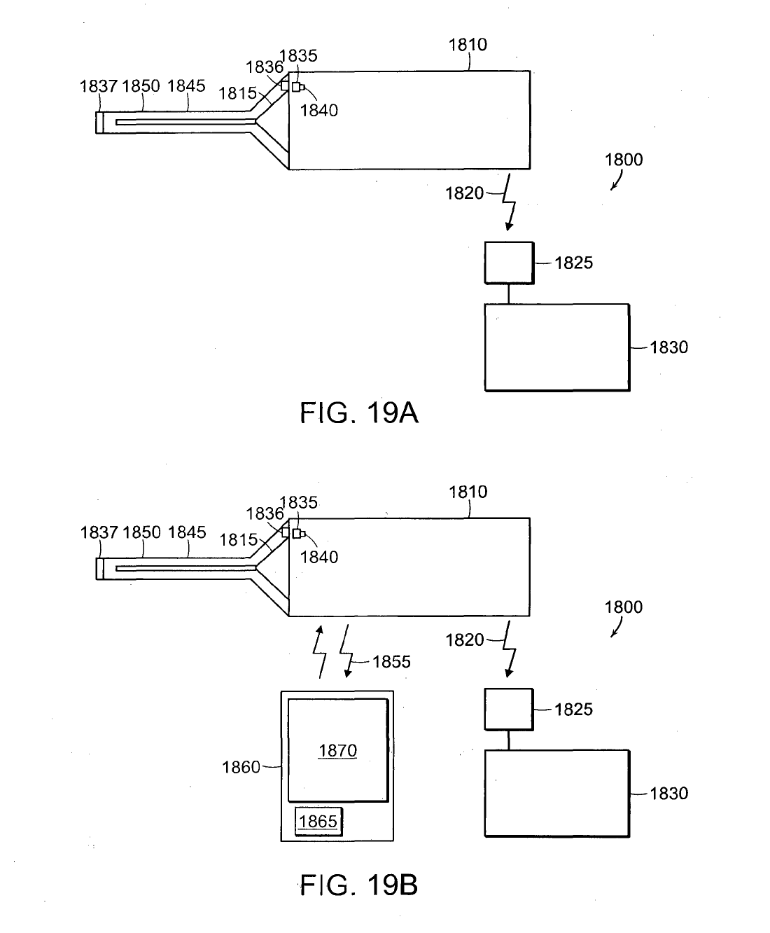

FIG. 18 shows a specific embodiment of how the camera head 1700 and peripherals can communicate in accordance with the invention. The camera head 1700 contains a serial peripheral interface (SPI) slave sensor board 1706 that communicates with a 3-CMOS image sensor 1708. The 3-CMOS sensor 1708 transmits and receives data and draws power from the transmitting/receiving unit 1764 of the video input board 1760. The transmitting/receiving unit 1764 is further in communication with the video DSR unit 1766 of the video input board 1760. The video input board 1760 also contains a microcomputer 1762. The video input board 1760 transmits data to and receives power from the CCU output board 1770. The data transmission can be, for example, in the form of serial data, HD or SD video data including video chromaticity information and video sync data, and clock speed. The video input board 1760 draws power (preferably 12VDC/2A) from the CCU output board 1770. The CCU output board 1770 contains a micro-computer and LCD touch screen front panel 1772. The micro-computer can communicate with users, user agents, or external devices such as computers using methods including, but not limited to, USB, Ethernet, or H.264 video. The Video DSP 1774 of the CCU output hoard 1770 is equipped to output DVI/HDMI or HD-SDI video to relevant devices. The CCU output board also contains a power unit 1776 and an LED power controller 1778. The LED power controller 1778 may be characterized by outputting constant current and by the capability to allow dimming of the LED. The camera head 1700 receives LED illumination from the LED light engine 1780. The LED light engine 1780 contains an LED illuminator 1784 that draws power (preferably 0-12 Amps at constant current, <5VDC) from the. LED power controller 1778. In turn, the LED illuminator 1784 powers a light fiber that feeds into the camera head 1700. The LED light engine 1780 also contains an LED heat sink fan 1782 that is powered by the power unit 1776 of the CCU output board 1770.

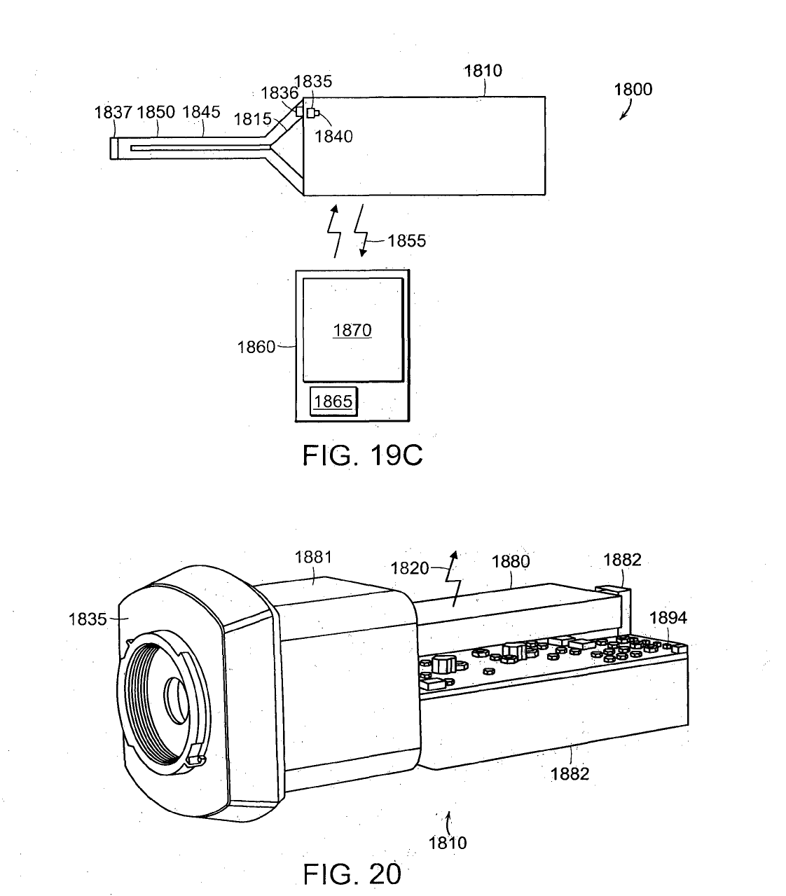

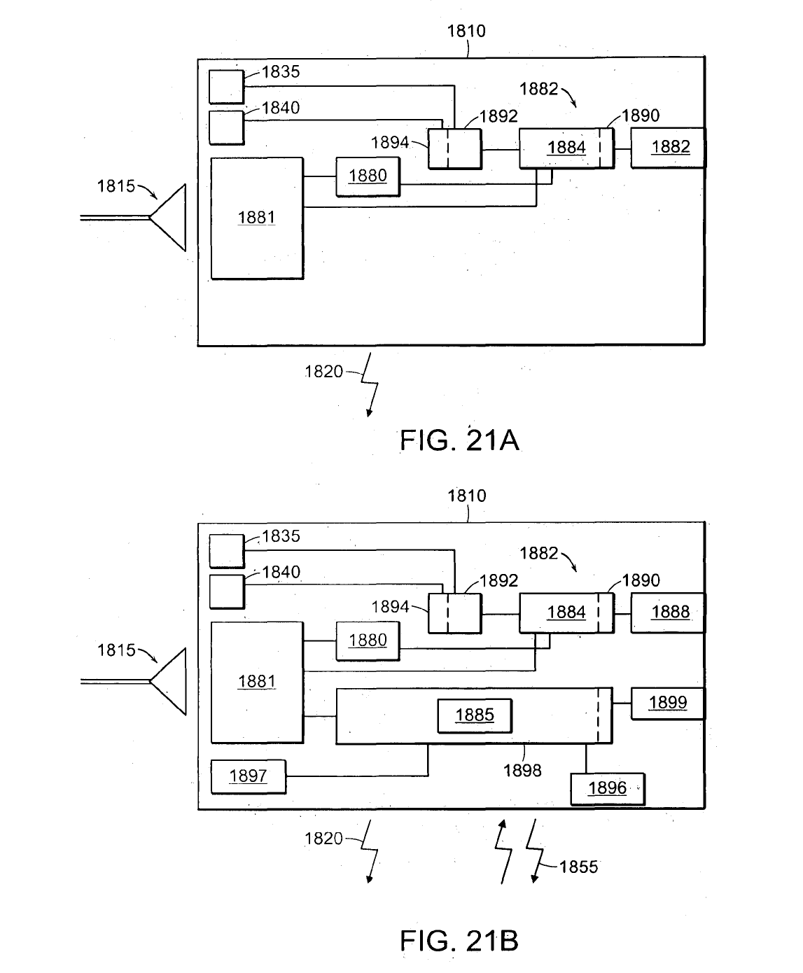

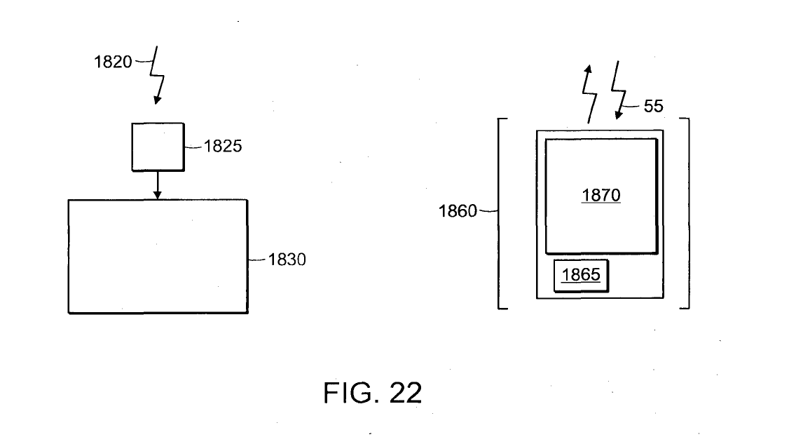

Turning more particularly to the drawings relating to a wireless endoscope handle, an embodiment of the wireless endoscopy system embodying the present invention is depicted generally at 1800 in FIG. 19A. There, the wireless endoscopy system 1800 includes a handheld camera handpiece 1810 that receives clinical imagery via an endoscope 1815. The camera handpiece 1810 wirelessly broadcasts radio frequency signals 1820 indicative of the clinical imagery that are received by a wireless video receiver 1825. The wireless video receiver 1825 is in communication with an electronic display 1830 that depicts the clinical imagery. An example video receiver 1825 is an ARIES Prime Digital Wireless HDMI Receiver manufactured by NYRIUS (Niagara Falls, ON, Canada). An example electronic display 1830 is the KDL-40EX523 LCD Digital Color TV manufactured by Sony (Japan). The camera handpiece 1810 may furthermore contain a source of illumination 1835 or a means of powering a source of illumination 1840 such as electrical contact plates or a connector. The system preferably operates at least at 10 frames per second and more preferably at 20 frames per second or faster. The time delay from a new image provided by the endoscope 1815 to its depiction at the electronic display 1830 is 0.25 seconds or less, and preferably is 0.2 seconds or less.

The first embodiment of the endoscopy system 1800 includes the camera handpiece 1810, the endoscope 1815, the receiver 1825 and display 1830, and a sterile barrier 1845 in the form of an illumination sheath 1850 that is discussed herein.

In some applications, it is permissible to sterilize the endoscope 1815 prior to each endoscopic imaging session. In other applications it is preferable to sheath the endoscope with a sterile barrier 1845. One type of sterile barrier 1845 is an illumination sheath 1850, similar to those described in U.S. Pat. No. 6,863,651 and U.S. patent application Pub. 2010/0217080, the entire contents of this patent and patent application being incorporated herein by reference. The sheath carries light from illumination source 1835 such that it exits the distal tip of the illumination sheath 1850.

Another type of sterile barrier 1845 does not require the handpiece 1810 to contain a source of illumination in that the sterile barrier 1845 can contain a source of illumination, for example an embedded illuminator 1836 in the proximal base, or a distal tip illuminator 1837 such as a millimeter-scale white light emitting diode (LED). In these cases, power can be coupled from the means of powering a source of illumination 1840. In all cases, the sterile barrier 1845 may or may not be disposable. The camera handpiece 1810 may perform other functions and has a variety of clinically and economically advantageous properties. FIG. 19B illustrates another embodiment of the endoscopy system 1800, in which the camera handpiece 1810 additionally broadcasts and optionally receives RF energy indicative of procedure data 1855, which includes one or more of: procedure imagery, procedure video, data corresponding to settings of the imager (white balance, enhancement coefficients, image compression data, patient information, illumination settings), or other image or non-image-related information. An endoscopy control unit 1860 executes an endoscopy software application 1865. The endoscopy software application 1865 performs the functions associated with the camera control unit (CCU) of a clinical endoscope, such as: image display, image and video storage, recording of patient identification, report generation, emailing and printing of procedure reports, and the setting of imaging and illumination parameters such as contrast enhancement, fiber edge visibility reduction, and the control of illumination 1835 or 1837. In one embodiment, a graphical user interface of the endoscopy software application 1865 appears on an electronic display 1870 of the endoscopy control unit 1860 and optionally also depicts the procedure imagery observed by the combined camera handpiece 1810 and endoscope 1815. Typically, the endoscopy control unit 1860 is a tablet computer utilizing any of a myriad of popular operating systems but can also be a computer in a non-tablet form factor such as a laptop or desktop computer and a corresponding display.

FIG. 19C illustrates a further embodiment of the endoscopy system 1800, which is similar to the embodiment of FIG. 19B except that the receiver 1825 and display 1830 are not present. That is, it illustrates a configuration in which the endoscopy control unit 1860 is sufficient to display the procedure imagery and video.

It will be understood in the field of endoscopy that elements of the endoscopy system 1800 can also be in communication with a wired or wireless network. This has utility, for example, for transmitting patient reports or diagnostic image and video data on electronic mail, to a picture archiving and communication system (PACS), or to a printer.

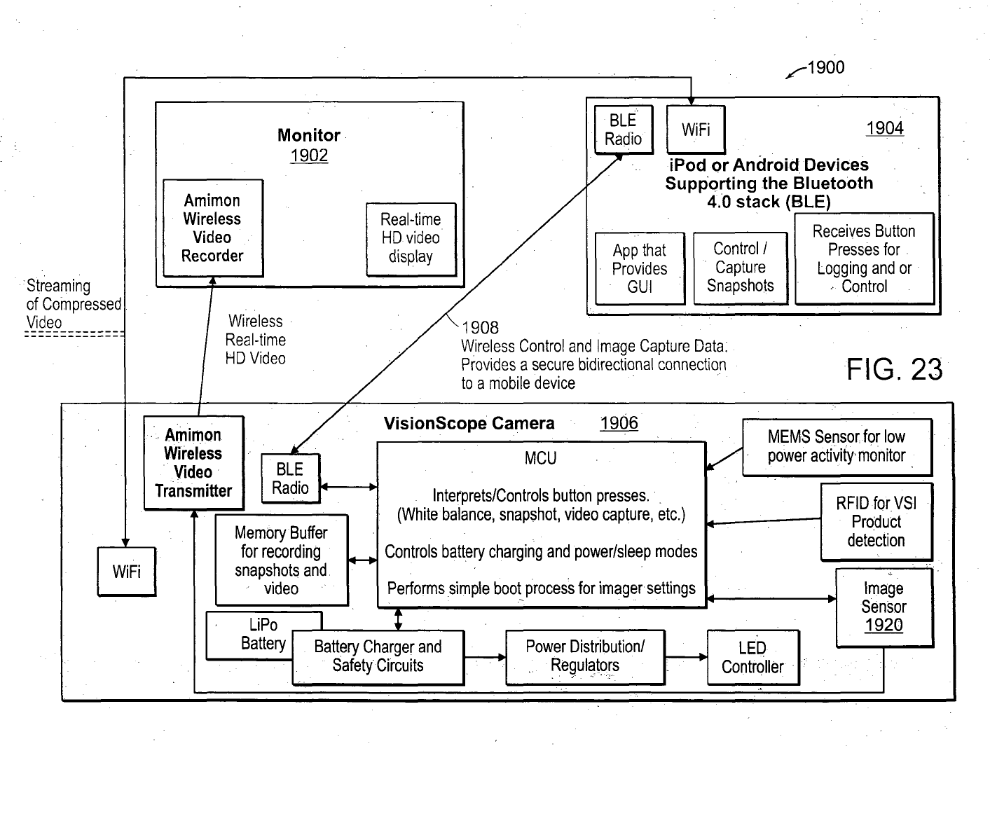

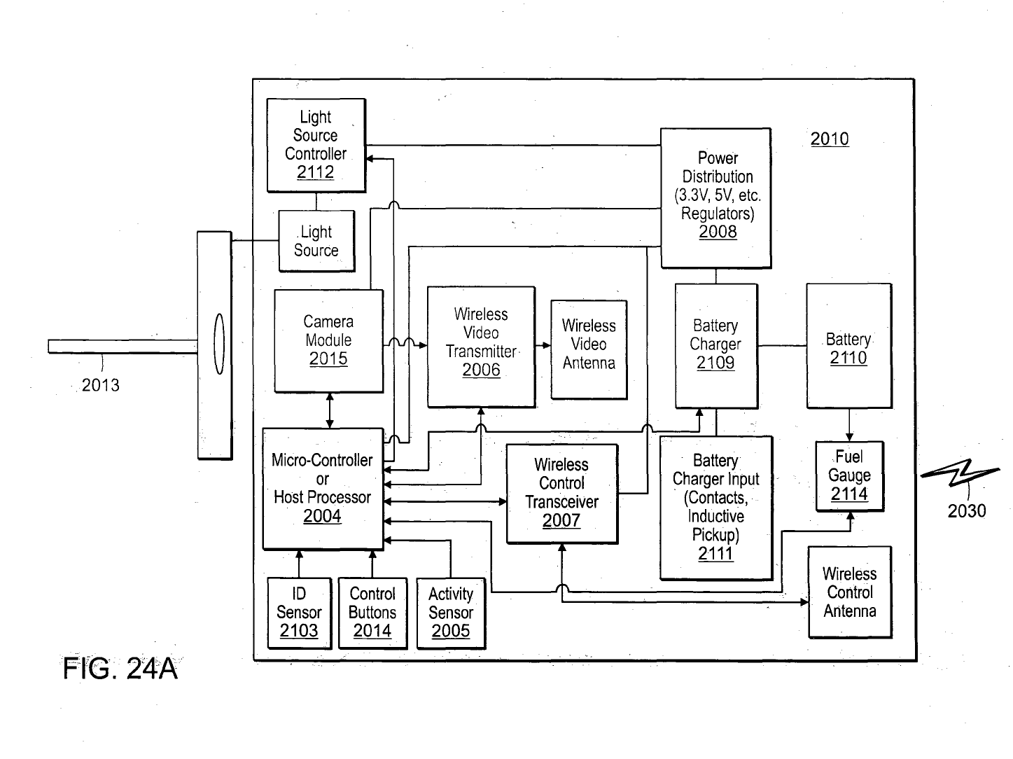

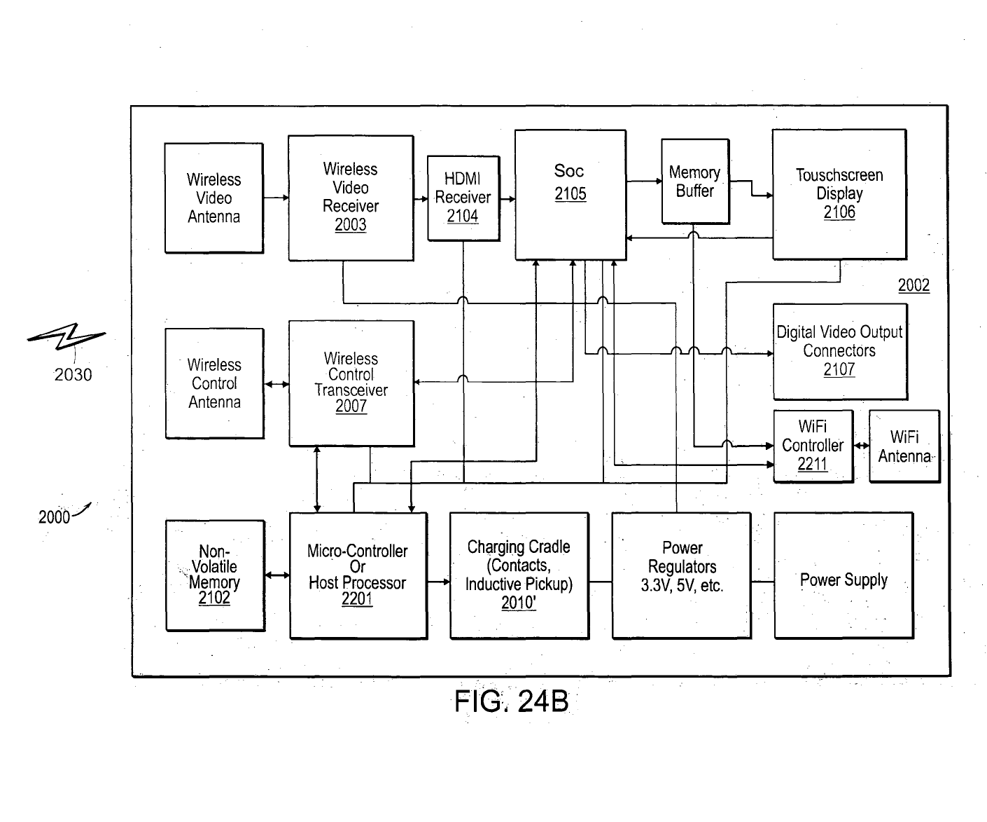

FIG. 20 illustrates a perspective view of the first embodiment of the camera handpiece 1810. FIG. 21A illustrates the camera handpiece 1810 and its components that may be used in various embodiments.

In a preferred embodiment, the camera handpiece 1810 receives optical energy corresponding to clinical imagery at an image capture electro-optical module, such as a digital image sensor module, model number STC-HD203DV, having an HD active pixel resolution of at least 1920×1080 (i.e., at least 2 million pixels or more) and a physical enclosure measuring at least 40 mm×40 mm×45.8 mm, manufactured by Sensor Technologies America, Inc. (Carrollton, Tex.), (i.e., between 60,000 mm³ and 200,000 mm³) and provides HDMI-formatted image data to a wireless video transmitter module 1880, such as the Nyrius ARIES Prime Digital Wireless HDMI Transmitter or Amimon Ltd. AMN 2120 or 3110 (Herzlia, Israel).

The wireless video transmitter module 1880 broadcasts the radio frequency signals 1820 indicative of the clinical imagery described in an earlier illustration. A power source 1882, for example a rechargeable battery 1884 or a single-use battery, and power electronics 1886, may receive electrical energy from a charger port 1888. The power electronics 1890 is of a configuration well-known to electrical engineers and may provide one or more current or voltage sources to one or more elements of the endoscopy system 1800. The power source 1882 generates one or more voltages or currents as required by the components of the camera handpiece 1810 and is connected to the wireless video transmitter module 1880, the image capture electro-optical module 1881, and the illumination source 1835 such as a white light-emitting diode (LED). For illustrative purposes, an LED power controller 1892 and a power controller for external coupling 1894 are also depicted, which can optionally be included in the handle.

It will be appreciated that the first embodiment incorporates a component-count that is greatly reduced compared to existing endoscopy systems and intentionally provides sufficient functionality to yield an endoscopy system when paired with the suitable wireless video receiver 1825 such as one using the Amimon AMI 2220 or 3210 chipsets and the electronic display 1830 such as the LCD display described earlier that preferably operates at HD resolution.

In other embodiments, as illustrated in FIG. 21B, the camera handpiece 1810 can have additional components and functionality and can be used with the endoscopy control unit 1860. The optional additional components of the other embodiments are described as follows:

The camera handpiece 1810 may include a camera controller and additional electronics 1898 in unidirectional or bidirectional communication with the image capture electro-optics module 1881. The camera controller and additional electronics 1898 may contain and perform processing and embedded memory 1885 functions of any of:

1. Sets imaging parameters by sending commands to the image capture electro-optics module, such as parameters corresponding to white balance, image enhancement, gamma correction, and exposure.

2. For an associated control panel 1896 having buttons or other physical user interface devices, interprets button presses corresponding for example to: “take snapshot,” “start/stop video capture,” or “perform white balance.”

3. Controls battery charging and power/sleep modes.

4. Performs boot process for imaging parameter settings.

5. Interprets the data generated by an auxiliary sensor 1897, for example “non-imaging” sensors such as an RFID or Hall Effect sensor, or a mechanical, thermal, fluidic, or acoustic sensor, or imaging sensors such as photodetectors of a variety of visible or non-visible wavelengths. For example, the electronics 1898 can generate various imager settings or broadcast identifier information that is based on whether the auxiliary sensor 1897 detects that the endoscope 1815 is made or is not made by a particular manufacturer, detects that the sterile barrier 1845 is or is not made by a particular manufacturer, or other useful functions. As an illustrative example, if the camera handpiece 1810 is paired with an endoscope 1815 that is made by a different manufacturer than that of the camera handpiece, and lacks an identifier such as an RFID tag, then the system does not detect that endoscope's model number or manufacturer and thus can be commanded to operate in a “default imaging” mode. If an endoscope of a commercially-approved manufacturer is used and does include a detectable visual, magnetic, RFID, or other identifier, then the system can be commanded to operate in an “optimized imaging” mode. These “default” and “optimized” imaging modes can be associated with particular settings for gamma, white balance, or other parameters. Likewise, other elements of the endoscopy system 1805 can have identifiers that are able to be sensed or are absent. Such other elements include the sterile barrier 1845.

6. Includes a memory for recording snapshots and video

7. Includes a MEMS sensor and interpretive algorithms to enable the camera handpiece to enter a mode of decreased power consumption if it is not moved within a specified period of time

8. Includes a Bluetooth Low Energy (BLE) module, WiFi module, or other wireless means that transmits and/or receives the procedure data 1855.

The camera controller and additional electronics 1894 may optionally be in communication with an electronic connector 1898 that transmits or receives one or more of: power, imagery, procedure settings, or other signals that may be useful for the endoscopy system 1800. The electronic connector 1898 can be associated with a physical seal or barrier such as a rubber cap as to enable sterilization of the camera handpiece 1810.

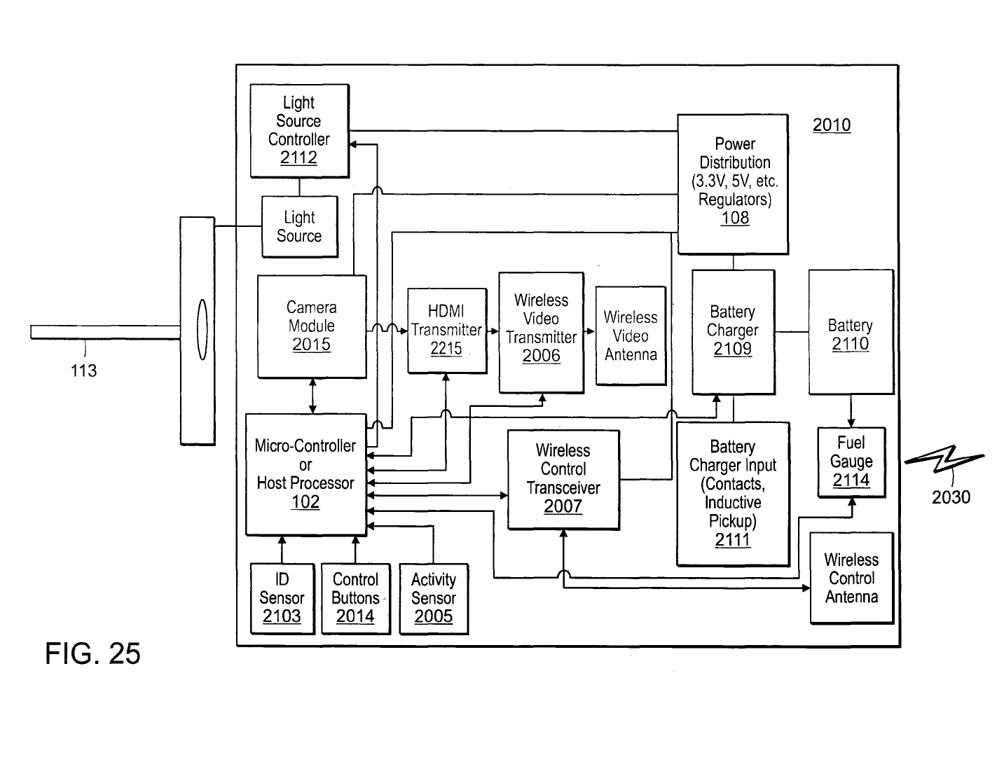

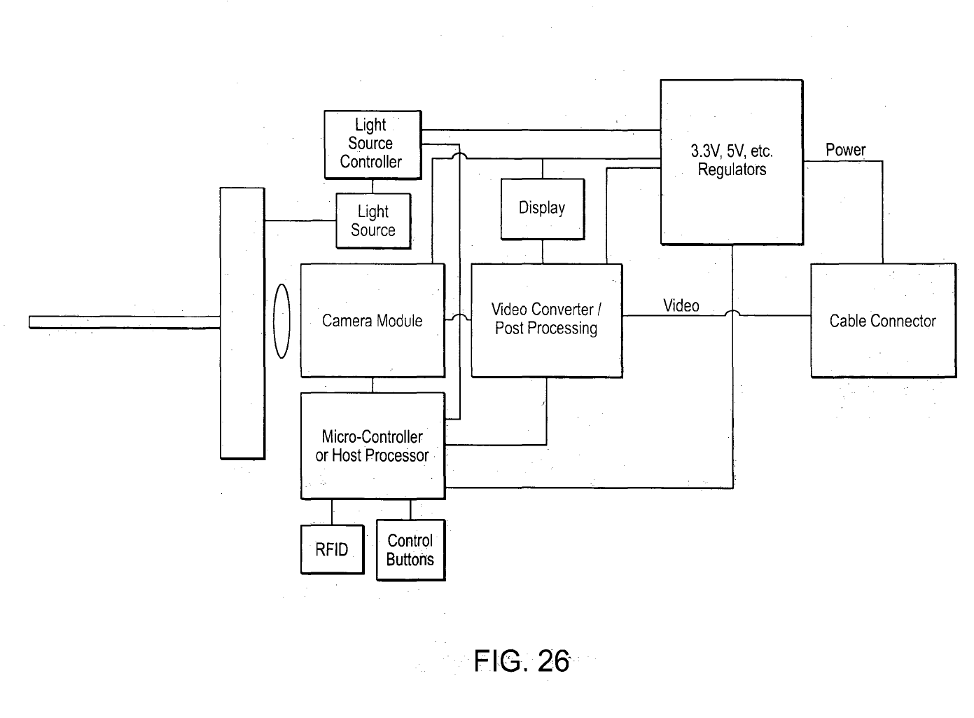

FIG. 22 illustrates the electronic display 1830 and the wireless video receiver 1825 of the first embodiment. It also illustrates the (optional) endoscopy control unit 1860 such as the tablet, with the electronic display 1870 and endoscopy software application associated with operation of a touchscreen processor 1865 that operates with a data processor in the tablet as described herein. FIG. 23 is a further illustration of the functional groups of preferred embodiments of the invention. The monitor 1902 can receive real time wireless video from the endoscope handle system 1906, while a separate link delivers a compressed video signal to the handheld display device 1904. A separate wireless bidirectional control connection 1908 can be used with the handheld device 1904, or, optionally with a separate dashboard control associated with monitor 1902. The handle 1906 is connected to the endoscope body as described previously. The image sensor 1920 can be located in the handle or at a distal end of the endoscope within the disposable sheath. For a system with a distally mounted image sensor, illumination can be with the annular fiber optic array as described herein, or with LEDs mounted with the sheath or the sensor chip or both.

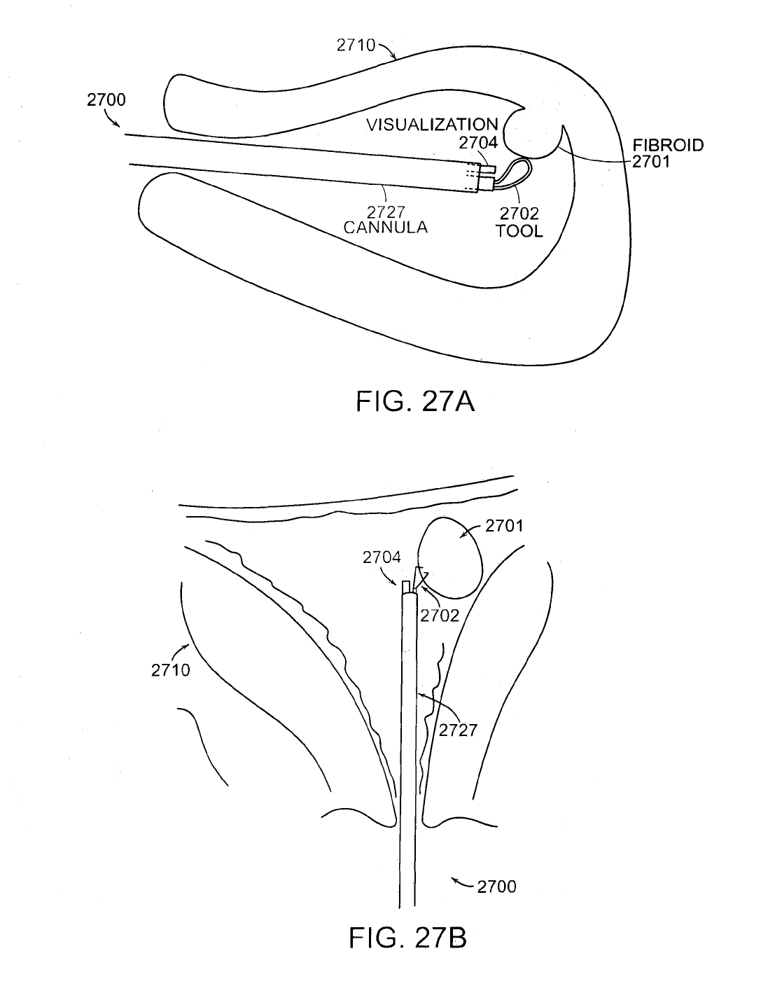

Illustrated in FIGS. 24A and 24B is a preferred embodiment of a wireless endoscopy system 2000. In the embodiment a wireless communications channel 2030 is inclusive of all wireless communications between a camera hand-piece or handle 2010 and a camera control unit (CCU) 2002 and may in practice be performed using one or more RF signals at one or more frequencies and bandwidths.

A camera module 2015, contained in the camera hand-piece 2010, receives optical energy from an illuminated scene that is focused onto the camera module's active elements in whole or in part by an endoscope 2013. The camera module 2015 translates the optical energy into electrical signals, and exports the electrical signals in a known format, such as the high definition multimedia interface (HDMI) video format. An example of this module is the STC-HD203DV from Sensor Technologies America, Inc.

The handheld camera device 2010 wirelessly transmits the HDMI video signal with low latency, preferably in real time, to a wireless video receiver 2003 via a wireless video transmitter 2006. The wireless video receiver 2003 is a component within the camera control unit 2002. An example of this wireless chipset is the AMN2120 or 3110 from Amimon Ltd.

In addition to the wireless video link described, a wireless control transceiver 2007 is used for relaying control signals between the camera device 2010 and the camera control unit 2002, for example control signals indicative of user inputs such as button-presses for snapshots or video recording. The wireless control transceiver 2007 is implemented using a protocol such as the Bluetooth Low Energy (BLE) protocol, for example, and is paired with a matching control transceiver 2012 in the camera control unit 2002. An example of a chipset that performs the functionality of the wireless control transceiver 2007 is the CC2541 from Texas Instruments, or the nRF51822 from Nordic Semiconductor. The wireless control transceiver 2007 sends and receives commands from a processing unit 2004, which can include a microcontroller such as those from the ARM family of microcontrollers.

In the first embodiment, the processing unit 2004 is in communication with, and processes signals from, several peripheral devices. The peripheral devices include one or more of: user control buttons 2014, an identification sensor 2103, an activity sensor 2005, a light source controller 2112, a battery charger 2109, and a power distribution unit 2008.

The identification sensor 2103 determines the type of endoscope 2013 or light guide that is attached to the camera hand-piece 2010. The processing unit 2004 sends the endoscope parameters to the camera control unit 2002 via the wireless control transceiver 2007. The camera control unit 2002 is then able to send camera module setup data, corresponding to the endoscope type, to the processing unit 2004 via the wireless control transceiver 2007. The camera module setup data is then sent to the camera module 2005 by the processing unit 2004. The camera module setup data is stored in a non-volatile memory 2102. The processing unit 2004 controls the power management in the camera hand-piece 2010 by enabling or disabling power circuits in the power distribution unit 2008.

The processing unit 2004 puts the camera hand-piece 2010 into a low power mode when activity has not been detected by an activity sensor 2005 after some time. The activity sensor 2005 can be any device from which product-use can be inferred, such as a MEMS-based accelerometer. The low power mode can alternatively be entered when a power gauge 2114, such as one manufactured by Maxim Integrated, detects that a battery 2110 is at a critically low level. The power gauge 2114 is connected to the processing unit 2004 and sends the status of the battery to the camera control unit 2002 via the wireless control transceiver 2007. The processing unit 2004 can also completely disable all power to the camera hand-piece 2010 when it has detected that the camera hand-piece 2010 has been placed into a charging cradle 2210 of the camera control unit 2002. In an embodiment in which the camera hand-piece is capable of being sterilized, the charging cradle 2210, and corresponding battery charger input 2111 contains a primary coil for the purpose of inductively charging the battery 2110 in the camera hand-piece 2010. In another embodiment where sterilization is not required, the charging cradle 2110 and corresponding battery charger input 2111 contain metal contacts for charging the battery 2110 in the camera hand-piece 2010. The touchscreen operates in response to a touch processor that is programmed to respond to a plurality of touch icons and touch gestures associated with specific operations features described herein.

Referring still to FIGS. 24A and 24B, more specifically to the camera control unit 2002, the video pipeline begins with the wireless video receiver 2003 which is in communication with the HDMI receiver 2104. The HDMI receiver 2104 converts the HDMI video into 24-bit pixel data which is used by a system-on-chip (SOC) 2105 for post processing of the video. The SOC 2105 can be any suitably-featured chip such as an FPGA with embedded processor, for example the Zynq-7000 from Xilinx. The post processed video is then sent to both the touchscreen display 2106 and to the digital video connectors 2107 which can be used for connecting external monitors to the camera control unit 2000. The SOC 2105 also has the capability to export compressed video data that can be streamed wirelessly to a tablet device using a Wi-Fi controller 2211 or similar device. In addition to post processing the video, the SOC 2105 also runs the application software. The camera control unit 2002 also contains a host processor 2201 for the control of peripherals, in particular, the charging cradle 2210. The embodiment of FIG. 24B can incorporate a touchscreen display into the handle, which can be used to manage computational methods, patient data entry, data and/or image storage and device usage data in the handle of the system. Alternatively, these functions can be shared with external processors and memory architecture, or can be conducted completely external to the handle.

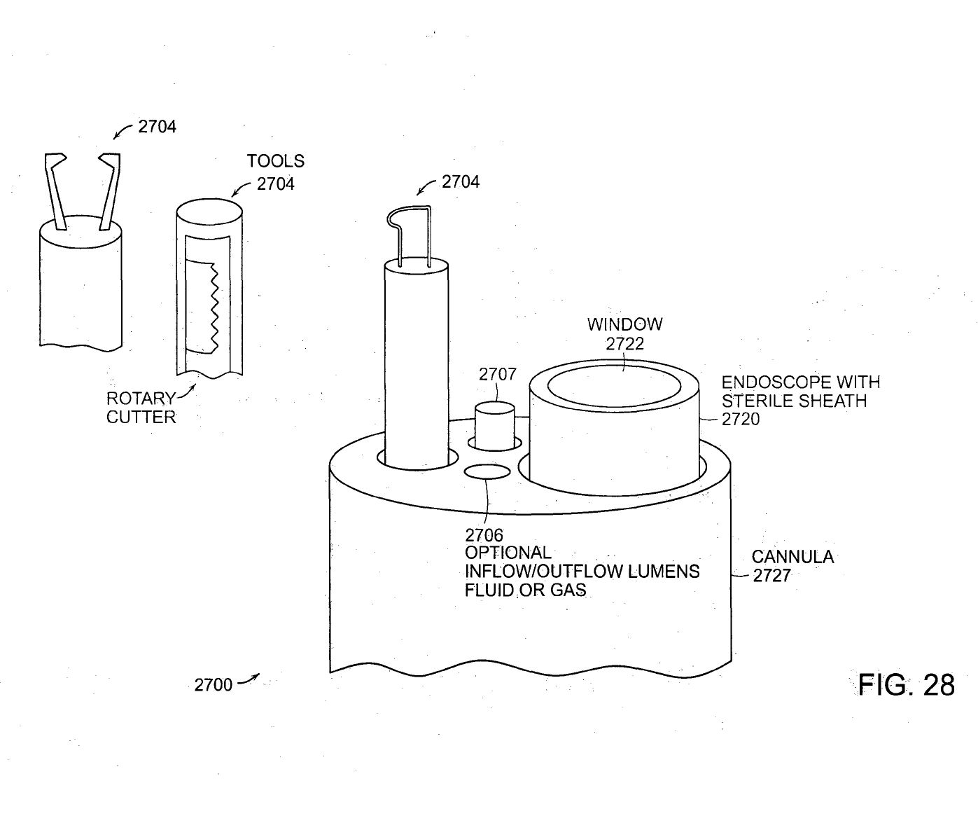

With reference to FIG. 25, the camera hand-piece 2010 contains an HDMI transmitter 2215. The HDMI transmitter 2215 is used in an embodiment where the camera module 2005 does not output HDMI formatted video. In this case, the camera module 2005 outputs pixel data that is processed and formatted by the HDMI transmitter 2215. All other components remain the same as in FIG. 24. It should be noted that in figures, the wireless channel 2030 can be replaced with a cable for a non-wireless system. Preferred embodiments of the camera module can provide a module output from any of the below sensors in a variety of formats, such as raw RGB data or HDMI: single chip CMOS or CCD with Bayer filter and a white LED with a fixed or variable constant current drive; or three chip CMOS or CCD with Trichroic prism (RGB splitter) and a white LED with a fixed or variable constant current drive; or single chip CMOS or CCD with no color filter wherein the light source can be pulsed RGB and, optionally, another wavelength (UV, IR, etc.) for performing other types of imaging.

Preferred embodiments can utilize different coupling from the handle to the endoscope to enable illumination; one or more LEDs coupled to fiber optics; one or more LEDs coupled to thin light guide file; one or more LEDs mounted in the tip of the endoscope; fiber optics or thin light guide film or HOE/DOE arranged on the outside diameter of elongated tube; the elongated tube itself can be a hollow tube with one end closed. The tube is made of light pipe material and the closed end is optically clear. The clear closed end and the light pipe tube can be extruded as one piece so it provides a barrier for the endoscope inside. This light source can be used for imaging through turbid media. In this case, the camera uses a polarizing filter as well.

The illumination can employ time-varying properties, such as one light source whose direction is modulated by a time-varying optical shutter or scanner (MEMS or diffractive) or multiple light sources with time-varying illumination.

To provide video with low latency, preferred embodiments can employ parallel-to-serial conversion camera module data (in the case where the module output is raw RGB and a cable is used to connect the camera to the camera control unit); direct HDMI from the camera module (can be used with or without a cable); cable harness for transmission of video data to a post processing unit in the absence of wireless; Orthogonal Frequency Division Multiplexing (OFDM) with multiple input multiple output wireless transmission of video (Amimon chip). In this case, the module data must be in HDMI format. If a camera module is used that has raw RGB output, there is an additional conversion from RGB to HDMI.

The display can comprise a small display integrated into the camera hand piece; a direct CCU to wireless external monitor; a display integrated into the camera control unit (CCU); a video streaming to a tablet; a head mounted display; or a specialized dock in the CCU capable of supporting a tablet (optionally with an adapter insert).

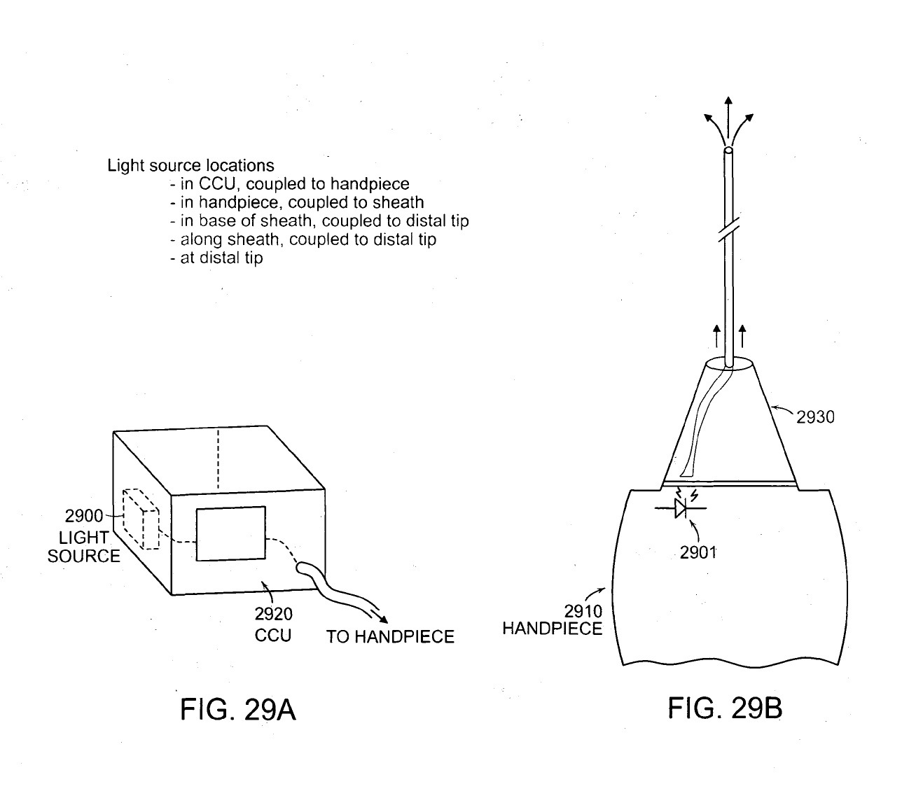

To provide systems for identification, control and patient data management, systems can use bluetooth low energy (BLE) for wireless button controls and for unit identification where BLE can also control power management; a secure BLE dongle on PC for upload/download of patient data; touchscreen on camera control unit for entering patient data and controlling user interface; keyboard for entering patient data and controlling user interface; WiFi-enabled camera control unit to connect to network for upload/download of patient data; integrated buttons for pump/insufflation control; ultrasound or optical time of flight distance measurement; camera unit can detect a compatible endoscope (or lack of) and can set image parameters accordingly; a sterile/cleanable cradle for holding a prepped camera; a charging cradle for one or more cameras; or inventory management: ability to track/record/communicate the usage of the disposables associated with the endoscopy system, and to make this accessible to the manufacturer in order to learn of usage rates and trigger manual or automated re-orders. Enabling technologies such as QR (or similar) Codes, or RFID tags utilizing near field communication (NFC) technology such as the integrated circuits available from NXP Semiconductor NV, on/in the disposables or their packaging, which can be sensed or imaged by an NFC scanner or other machine reader in the camera handpiece or the CCU. FIG. 26 illustrates an embodiment including an RFID scanner within the handle along with a display to view images.

Image processing can employ software modules for image distortion correction; 2D/3D object measurement regardless of object distance; or utilization of computational photography techniques to provide enhanced diagnostic capabilities to the clinician. For example: H.-Y. Wu et al, “Eulerian Video Magnification for Revealing Subtle Changes in the World,” (SIGGRAPH 2012) and Coded aperture (a patterned occluder within the aperture of the camera lens) for recording all-focus images. With the proper image processing, it might give the ability to autofocus or selectively focus without a varifocal lens. E.g.: A. Levin et al, “Image and Depth from a Conventional Camera with a Coded Aperture,” (SIGGRAPH 2007). A digital zoom function can also be utilized. Optical systems can include a varifocal lens operated by ultrasound; or a varifocal lens (miniature motor).

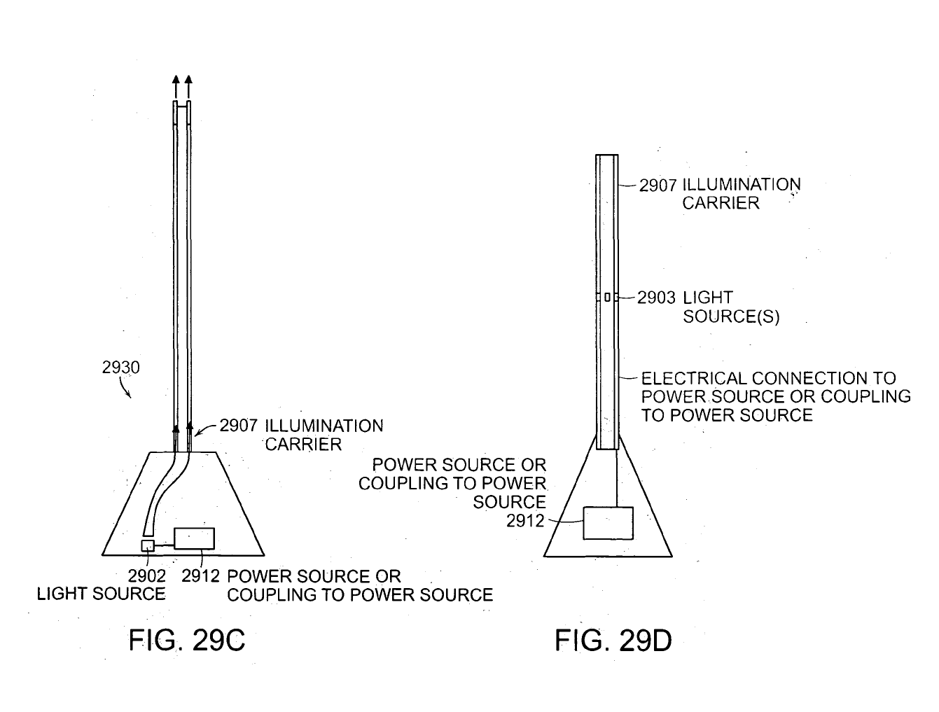

Turning to FIGS. 27A and 27B, a surgical device 2700 is shown that can help diagnose and treat ailments in a uterus 2710 or uterine wall. The surgical device 2700 can include a cannula 2727 through which various surgical tools and visualization devices may pass. In some embodiments, surgical tools and visualization devices are integrated directly into the cannula or a sheath. In some embodiments, the surgical device 2700 does not contain a cannula. The surgical device 2700 can be used to perform minimally-invasive surgical procedures including, but not limited to, biopsy, polypectomy, myomectomy, hysterectomy, and visual dilation and curettage.

Each year, millions of women seek treatment for fibroids. Fibroids are noncancerous growths that appear in the uterine wall. For moderate to severe cases of fibroids, a surgical approach is recommended. In accordance with various embodiments, surgical devices 2700 of the present invention can be used to diagnose and treat suspicious lesions 2701 including fibroids in the uterus 2700 as shown in FIGS. 27A and B. To perform a procedure, the surgical device 2700 can be inserted through the cervix or may be inserted through a vaginal or abdominal incision. A visualization device 2704 such as an endoscope is inserted through a lumen in the cannula 2727. A surgical tool 2702 may also be inserted through a lumen in the cannula. In some embodiments, the visualization device 2704 and surgical tool 2702 are inserted in separate and neighboring lumens in the cannula 2727. As the tool 2702 interacts with the suspicious lesion 2701, a user can visualize the region of interest in real time using the visualization device 2704.

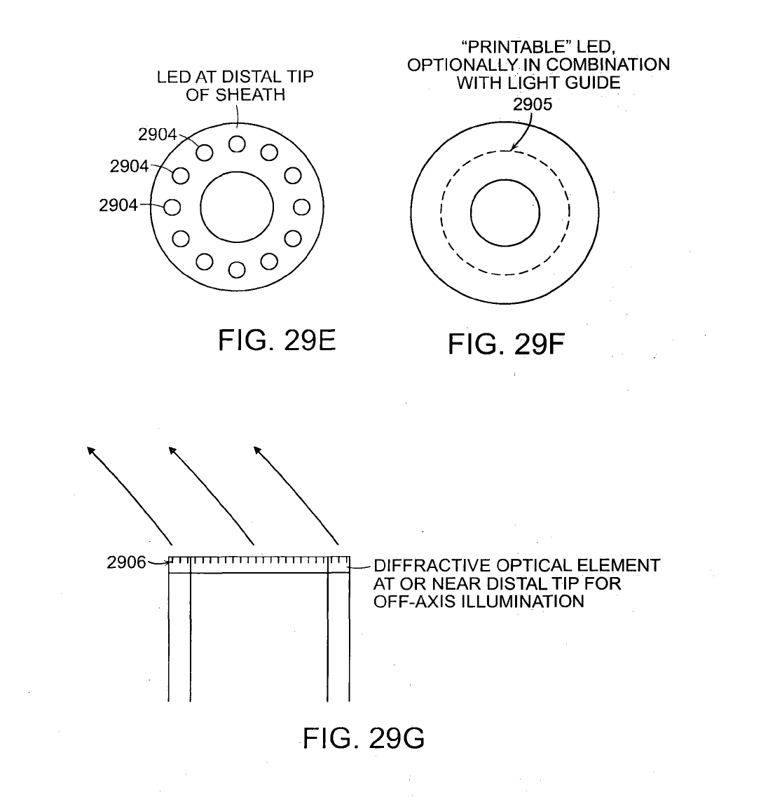

FIG. 28 shows a magnified view of the distal tip of the surgical device 2700. As described above, the device 2700 includes a cannula 2727 that may contain one or more lumens. A tool 2704 can pass through a lumen of the cannula 2727. In some embodiments, the tool 2704 may be completely withdrawn from the surgical device 2700. The visualization device 2720 can include a window at the distal tip and a sterile sheath as set forth in the embodiments described in the present application. An endoscope can rest within the sterile sheath, and the entire visualization device 2720 may be advanced or withdrawn within the lumen of the cannula 2727. Alternatively, the tool(s) 2704 and visualization device 2720 may be integrated directly with the cannula 2727. The cannula 2727, tool 2704, and visualization device 2720 can be rigid, semi-rigid, steerable, or non-rigid. In some embodiments, the cannula 2727 can include additional inflow or outflow lumens 2706 to add or withdraw fluids or gases from the distal tip of the cannula 2727. The inflow or outflow lumens 2706 may be used in conjunction with a tube 2707 that may be inserted through the lumen 2706 to deliver fluids and gases to a region of interest. The tube 2707 can also beneficially prevent fouling or clogging of the lumen 2706. In accordance with various embodiments, the lumen 2706 for inflow/outflow, the lumen for the tool 2704, and the lumen for the visualization device 2704 may be separate or combined.

The surgical tool 2704 may be one or more from a range of tools including but not limited to forceps, hot biopsy forceps, snares, electrosurgical tools, cutting tools, abrading tools, lasers, rotary cutters, scissors, scalpels, radio-frequency (RF) tools, morcellators, or any other tool suitable to meet application-specific requirements. In some embodiments, the tool 2704 can be completely withdrawn from the cannula 2727 and later reinserted to allow, for example, for obtaining multiple biopsy samples.

The visualization device 2704 can include an endoscope inside a sterile sheath. The endoscope may be similar to that described above with respect to FIG. 1. For example, the endoscope may include optical fibers collected into a bundle in a tightly spaced arrangement. The window 2722 can be attached to the sterile sheath 2720 to produce a fluid-tight seal. In some embodiments, the sterile sheath 2720 may be made of a polymeric material.

Ultraviolet light-curable materials may also be used in conjunction with the surgical device 2700 or other devices and methods as described generally herein according to the present invention. Ultraviolet light-curable (or UV-curable) materials may be used to perform certain types of tissue repairs or may strengthen or fixate a tissue. In accordance with various embodiments, UV-curable materials can include, but are not limited to, SpeedMask® (Dymax Corporation, Torrington, Conn.) and Rose Bengal alone or in conjunction with other materials such as chitosan. In some embodiments, the UV-curable materials may be injected into a joint through a single-or dual-port cannula of the present devices. For example, a tube 2707 may be inserted through a lumen 2706, and the visualization device 2704 can observe the distal end of the tube 2707 as it releases material in a targeted manner in the region of interest. To cure ultraviolet light-reactive materials, a source of ultraviolet light is needed. In some embodiments, UV light may be delivered through the optical fibers of the device, may come from other emitters near the distal end of the device, or may be delivered via a separate probe. In some embodiments, the UV-curable materials can be mixed with fibrin glue or other mixable or embeddable materials to help with meniscal repair and repair of osteochondral defects. In an exemplary procedure, the UV-curable material can be injected into a meniscal tear or chondral defect, and subsequent exposure to UV light hardens the material to form a bond. The hardness and/or porosity of the material can have a range of values depending on whether the location of the repair is, for example, meniscus, articular cartilage, or the rotator cuff and the need for additional therapeutic agents that may be inserted with the material into the body.

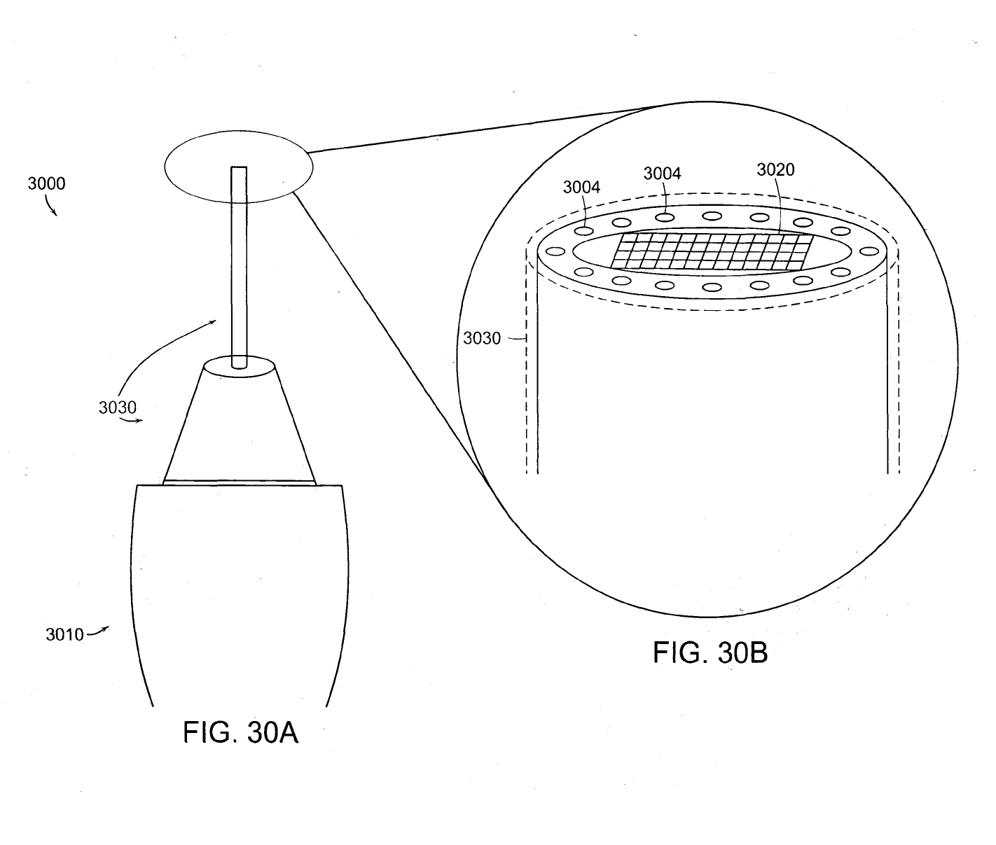

The light source to provide illumination at the distal end of a visualization device may be placed in a variety of locations. Several exemplary embodiments are described herein. In the embodiment of FIG. 29A, the light source 2900 may be located within a camera control unit similar to that described with reference to FIG. 17. Light from the light source 2900 is carried out of the camera control unit 2920 and coupled through the handpiece 2910 to the distal tip of the sheath 2930. In the embodiment of FIG. 29B, the light source 2901 resides within the handpiece 2910. Power for the light source 2901 can be provided by a battery within the camera control unit 2920, handpiece 2910, sheath 2930, illumination carrier, cannula, or endoscope or may be provided by an external source. The battery or external power source can be in direct electrical contact or may be inductively coupled to the light source 2902. The light from the light source 2901 can be coupled to the distal tip of the sheath 2930. In some embodiments, the light source 2901 can be an LED source.

In the embodiment of FIG. 29C, the light source 2902 is contained within the base of the sheath 2930. A battery 2912 to power the light source 2902 may be provided in the base of the sheath 2930 or in other places within the device as described above with reference to FIG. 29B. Alternatively, the light source 2902 may be powered using an external source. The battery or external power source can be in direct electrical contact or may be inductively coupled to the light source 2902. Light from the light source 2902 may be delivered to the distal tip of the sheath 2930 using an illumination carrier 2907. The illumination carrier 2907 can be arranged as an annulus about the sheath 2930 in some embodiments. In the embodiment of FIG. 29D, the light source 2903 is placed at a point within an illumination carrier 2907 between the distal end and base of the sheath. An electrical connection can pass within the illumination carrier 2907 to a power source 2912 residing in the base of the sheath 2930 or other places within the device as described above with reference to FIG. 29B. In some embodiments, the electrical connection can pass out of the sheath 2930 or into the handpiece 2910 to connect to a battery or external power source. Alternatively, the battery or external power source can be inductively coupled to the light source 2902.

In the embodiment of FIG. 29E, the light source may include LEDs 2904 mounted directly at the distal tip of the sheath 2930. In the embodiment of FIG. 29F, the LEDs 2905 may be printed at the distal tip of the sheath 2930. In some embodiments, the LEDs can optionally be combined with a light guide as described previously with respect to other embodiments. In accordance with various embodiments, the LEDs 2904, 2905 may be individual components mounted to the distal tip or integral components fabricated with or printed onto the sheath 2930. The device can comprise an LED ring illuminator having a diameter of 4 mm or less, or in certain preferred embodiments having a diameter of 3 mm or less, and preferably 2 mm or less. The LEDs can comprise an annular array with the angular spacing between LED elements being 30 degrees or less. Those skilled in the art will appreciate that the LED elements do not need to be symmetrically arranged and/or may be placed in a non-annular distribution. Preferably, at least 12 LEDs are used to form an annular ring. Although a certain number of LEDs is depicted in FIG. 29E, those skilled in the art will appreciate that any number of LEDs may be used including, for example, smaller numbers such as two or four LEDs. Also, the spaced LEDs can use a diffuser extending over the LED elements to diffuse the emitted light. A battery to power the LEDs 2904 may be located at various locations with the device as described above with reference to FIG. 29B or may be powered by an external source.

The distal tip of the sheath 2930 may also contain a diffractive optical element 2906 in accordance with various embodiments and as shown in FIG. 29G. The diffractive optical element 2906 may be used to enable off-axis illumination and light collection. In certain embodiments, the diffractive optical element 2906 may be used to enhance illumination or observation near the end of an endoscope, or tool, or a region of interest. In various embodiments, the diffractive optical element 2906 can be a holographic optical element (HOE) or diffraction grating.

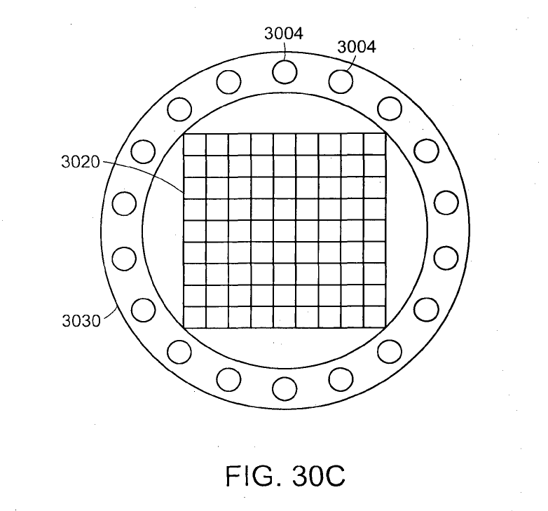

In accordance with various embodiments, a surgical device 3000 shown in FIGS. 30A-30C may include a sensor 3020 at the distal tip of a sheath 3030. The device 3000 can include a handpiece 3010 and a sheath 3030 including a base and an extended portion as described above with respect to previous embodiments. In some embodiments, the distal tip of the sheath 3030 may include a window that is sealed to prevent ingress of fluid or other material as described previously with respect to other embodiments. The sensor 3020 can be located behind the window. The diameter of the extended portion of the sheath 3030 of the device 3000 may be 3 mm, or preferably less than 2 mm. In some embodiments, the diameter of the extended portion of the sheath 3030 of the device 3000 may be less than 1 mm.

In an exemplary embodiment, the sensor 3020 can be a charge-coupled device (CCD) or CMOS array. The sensor is made up of an array of pixels. The sensor 3020 can have an individual pixel size of 3 microns or, more preferably, 2 microns or less. In some embodiments, the pixel size of a CCD sensor can be 1 micron. In some embodiments, the sensor 3020 can include an array size between 200×200 (40,000) and 500×500 (250,000) pixels.

The device 3000 can include LEDs 3004 mounted at the distal tip of the sheath 3030 to illuminate an area of interest. The sheath 3030 can include a distal window to seal the imaging device within the sheath 3030 as previously described. The LEDs 3004 may be similar to those described above with respect to previous embodiments. In some embodiments, selective filters may be mounted inside or outside the window at the distal tip of the sheath 3030. These filters can include, but are not limited to, polarization filters, wavelength filters, and anti-reflective filters. In various embodiments, the LEDs 3004 may be connected and arranged to be able to power on individually or simultaneously. In some embodiments, the LEDs 3004 may be illuminated individually in a sequential pattern to generate images having differential shadow patterns for imaging and quantifying topological features of a tissue. An analysis of the differential shadow patterns can provide elevation or topological information for each pixel of an image. Alternative techniques for topographical mapping in addition to depth-from-defocus include those known to people skilled in the art such as structure-from-motion and structured light. The resulting 2D and 3D imagery can be analyzed using other known techniques to classify anatomic features as healthy or damaged or as needing repair, removal, or further diagnosis, or being responsive to various wavelengths with or without fluorescent dyes. As a result of this analysis, the system can determine sizes and shapes of anatomic features and can visually differentiate a region using an outline, shading, or other known techniques of graphical or alphanumeric tagging.

With certain details and embodiments of the present invention for endoscopic systems described herein, including wireless endoscopy systems, it will be appreciated by one skilled in the art that changes and additions could be made thereto without deviating from the spirit or scope of the invention.

The attached claims shall be deemed to include equivalent constructions insofar as they do not depart from the spirit and scope of the invention. It must be further noted that a plurality of the following claims may express certain elements as means for performing a specific function, at times without the recital of structure or material and any such claims should be construed to cover not only the corresponding structure and material expressly described in this specification but also all equivalents thereof.

1. An endoscope system comprising:
an endoscope handle in communication with an external display device;
a visualization device for imaging a region of interest within a body;
a surgical tool;
a tubular sheath having a diameter of 3 mm or less that receives the visualization device; and
a cannula system to introduce the surgical tool and the tubular sheath into a joint cavity.

2. The system of claim 1 wherein the tubular sheath comprises a tubular body having an inner tube.

3. The system of claim 1, wherein the tubular sheath has a diameter of 2 mm or less.

4. The system of claim 1, wherein the surgical tool comprises an arthroscopic tool.

5. The system of claim 4, wherein the arthroscopic tool comprises at least one of a cutting tool, abrading tool, mechanized rotary cutter, electrosurgical tool, laser, scalpel, forceps, snare, morcellator, RF cutting element, or electrically powered tool.

6. The system of claim 1, wherein the visualization device comprises a tube with a plurality of optical fibers, the tube being insertable within the tubular sheath having a straight or curved shape.

7. (canceled)

8. The system of claim 1, wherein the cannula system comprises a single cannula body having a first cannula channel and a second cannula channel.

9. The system of claim 1, wherein a distal end of the surgical tool is 4 mm in size or less.

10. (canceled)

11. The system of claim 1, wherein the tubular sheath has a distal optical assembly to image a field of view.

12. (canceled)

13. (canceled)

14. The system of claim 1, wherein a lumen of a cannula is used to deliver an ultraviolet light-curable material to a region of interest within a patient.

15. (canceled)

16. The system of claim 1, wherein the surgical tool is configured to bend in a predetermined manner upon protrusion from a cannula.

17-20. (canceled)

21. The system of claim 1, wherein the endoscope handle comprises a power source, a power regulation circuit, a video transmitter and a control transceiver.

22. (canceled)

23. The system of claim 1, wherein the tubular sheath comprises one or more light-emitting diodes (LEDs) at a distal tip.

24-35. (cancelled)

36. The system of claim 1, wherein the endoscope handle is configured to operate with one or more imaging sensors that are optically coupled to a fiber optic imaging bundle that extends within an imaging tube.

37. (canceled)

38. (canceled)

39. The system of claim 1, wherein the surgical tool is operatively configured to perform at least one of a biopsy, a myomectomy, a polypectomy, a hysterectomy, or a visual dilation and curettage procedure.

40. (canceled)

41. The system of claim 1, further comprising a source of illumination.

42. The system of claim 41 wherein the source of illumination is located in the handle.

43-47. (canceled)

48. The system of claim 1, wherein a first cannula is inserted through a first entry point and a second cannula is inserted through a second entry point separated from the first entry point.

49. The system of claim 1, further comprising a tube passing through a lumen in a cannula to deliver liquids or gases to a region of interest, the tube being removable from the lumen.

50. An endoscope system comprising:
an endoscope handle in communication with an external display device;
an endoscope including a tubular visualization device; and
a tubular sheath having a diameter of 3 mm or less, the sheath having a distal optical assembly to image a field of view, the sheath further comprising an annular array of optical fibers,
wherein an angle of view of the visualization device is offset from an insertion axis of the endoscope device.

51. The system of claim 50, wherein the system further comprises an arthroscopic tool operatively configured for insertion through a cannula channel.

52. (canceled)

53. The system of claim 50, wherein the angle of view is defined by an angle relative to the insertion axis in a range of 5-45 degrees.

54. (canceled)

55. The system of claim 50, wherein the tubular sheath has a diameter of 2 mm or less.

56-60. (canceled)

61. The system of claim 51 wherein the arthroscopic tool is inserted through a first cannula channel and the tubular sheath is inserted through a second cannula channel.

62. (canceled)

63. A method for arthroscopic surgery comprising:
inserting a distal end of an endoscope system through a first cannula channel into a body cavity, the endoscopic system including a tubular endoscopic device and a tubular sheath having a diameter of 3 mm or less;
inserting a surgical tool through a second cannula channel; and
viewing a surgical procedure performed with the surgical tool using the endoscope system.

64. The method of claim 63 further comprising surgically treating a meniscus within a joint of a patient.

65. (canceled)

66. The method of claim 63 further comprising inserting the endoscope through a first cannula at a first surgical access position and inserting a surgical tool through a second cannula that has a diameter of 2 mm or less.

67. The method of claim 63 further comprising a single cannula body that includes the first cannula channel and the second cannula channel.

68. (canceled)

69. The method of claim 63 further comprising transmitting images using a wireless video transmission connection.

70-84. (canceled)
